(19) United States
(12) Patent Application Publication
(10) Pub. No.: US 2015/0246908 A1
(43) Pub. Date: Sep. 3, 2015

(54) KLF5 MODULATORS

(71) Applicants: The Scripps Research Institute, La Jolla, CA (US); The Research Foundation for the State University New York, Albany, NY (US)

(72) Inventors: Thomas D. Bannister, Palm Beach Gardens, FL (US); Yuanjun He, Palm Beach Gardens, FL (US); Agnieszka Bialkowska, East Setauket, NY (US); Vincent Yang, East Setauket, NY (US); Melissa Crisp, La Jolla, CA (US); Peter Hodder, Jupiter, FL (US)

(21) Appl. No.: 14/417,396

(22) PCT Filed: Jul. 26, 2013
(86) PCT No.: PCT/US13/52272

§ 371 Date: Jan. 26, 2015

Related U.S. Application Data
(60) Provisional application No. 61/676,051, filed on Jul. 26, 2012.

(51) Int. Cl. C07D 417/12; C07D 333/48; C07D 409/12; C07D 335/00

(52) U.S. Cl. CPC C07D 417/12; C07D 335/00; C07D 333/48; C07D 409/12

(57) ABSTRACT

The invention provides potent inhibitors of small molecule inhibitors of Krüppel-like factor 5 (KLF5) expression. Compounds of the invention are small molecule inhibitors of KLF5 expression which can be effective delay or prevent colon cancer onset, halt growth of existing tumors, and/or decrease reoccurrence. The compounds can be effective vs. many tumor types whose progression is in part mediated by KLF5, including colorectal cancers. Lowering KLF5 levels with an inhibitor of this invention may also impact other disease states, including diabetes, obesity, lipid homeostasis, cardiovascular disease, and arthritis.

### CROSS-REFERENCE TO RELATED APPLICATIONS

This application claims the priority of U.S. provisional application Ser. No. 61/676,051, filed Jul. 26, 2012, which is incorporated herein by reference in its entirety.

### STATEMENT OF GOVERNMENT SUPPORT

This invention was made with government support under grant numbers 1 U54 MH084512-01 and DA026215-01, awarded by the National Institutes of Health. The U.S. government has certain rights in the invention.

### BACKGROUND

Transcription factors are regulatory DNA-binding proteins that control rate and frequency of gene expression¹. Many disease states are associated with aberrant transcription factor levels², including certain aggressive malignancies wherein transcription factors typically scarce or absent in normal cells are abundant. Malignant epithelial cells in intestinal crypts have elevated levels of the zinc finger-containing transcription factor Krüppel-like factor 5 (KLF5), also called intestinal-enriched Krüppel-like factor (IKLF), which binds to GC-rich sequences in promoters of numerous genes^{3,4} including cyclin D1,⁵ cyclin B1/Cdc2,⁵ and integrin-linked kinase,⁶ to mediate certain transforming effects of oncogenic HRAS.^{7,8}

Oncogenic KRAS mutations are present in ˜40% of colorectal cancers and its oncogenic mutational effects are in part mediated by KLF5, as shown by a study wherein polyp formation of mutant KRAS-associated cells in vivo was found to be KLF5-driven: genetic reduction of KLF5 in transgenic mice resulted in a significant reduction in intestinal tumor formation in mice strains harboring a germline mutation in the murine Apc gene and in a strain having combined Apc and KRAS mutations (reduction in intestinal polyp formation was 96 and 92%, respectively).^{9,10} Analysis of KLF5 expression patterns in tumors and the finding that ectopic expression of KLF5 lead to increased cell proliferation and anchorage-independent growth of cultured intestinal epithelial cells^{11,12} further support the contention that KLF5 plays a role in KRAS-driven colon cancer pathogenesis. Selective inhibitors of KLF5 production may help elucidate KLF5's role as a regulator of proliferation and a mediator of tumor formation in the intestinal epithelium and may also be effective chemotherapeutics.

KLF5 may play a role in the progression of other tumor types as well: in an epidemiological study breast cancer patients with high KLF5 levels were found to have shorter disease-free survival and lower overall survival¹³. KLF5 over-expression is thought to also impact other disease states: KLF5 promotes adipogenesis and adipocyte differentiation by transactivation of PPARgamma2,¹⁴ KLF5 also appears to promote cartilage degradation by the up-regulation of MMP-9.¹⁵

### SUMMARY

The present invention is directed, in various embodiments, to small molecule inhibitors of Krüppel-like factor 5 (KLF5) expression, to methods of using the inhibitors, and to methods of making the inhibitors. In various embodiments, the invention provides a small molecule inhibitor of KLF5 expression which can effectively delay or prevent colon cancer onset, halt growth of existing tumors, and/or decrease reoccurrence. The compound can be effective vs. many tumor types whose progression is in part mediated by KLF5, including colorectal cancers. Lowering KLF5 levels with an inhibitor of this invention may also impact other disease states, including diabetes, obesity, lipid homeostasis, cardiovascular disease, and arthritis. The inventors here have used an ultra-high throughput screening (uHTS) approach, follow-up mechanistic studies, and medicinal chemistry efforts aimed to enhance potency, selectivity, lead-like and drug-like properties¹⁶⁻²⁰ to discover potent inhibitors of KLF5 expression.

The invention provides, in various embodiments, a KLF5 expression-inhibitory compound of formula (I)

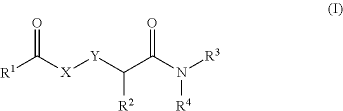

wherein

each R is independently H or (C₁-C₆)alkyl;

R¹ is aryl, arylalkenyl, heteroaryl, or heteroarylalkenyl, wherein any aryl or heteroaryl of R¹ is optionally mono- or independently multi-substituted with J;

X═CHR, O, or NR₂;

Y═CHR or absent;

R² is H, (C₁-C₆)alkyl, hydroxy(C₁-C₆)alkyl, (C₁-C₆)alkylNR₂, aryl, benzyl, p-hydroxybenzyl, (C₁-C₆)alkyl-C(═O)NR₂, (C₁-C₆)alkyl-C(═O)OR, or heteroaryl;

R³═H, (C₁-C₆)alkyl; (C₃-C₁₀)cycloalkyl, (C₁-C₆)alkylNR₂, (C₁-C₆)alkyl(C₃-C₁₀)cycloalkyl, or (C₁-C₆)alkyl-(3- to 10-membered heterocyclyl);

R⁴ is a 5-7 membered heterocyclyl comprising at least one C(═O)NR group or SO₂ group, optionally further comprising an additional NR group

J is halo, CF₃, OCF₃, CH₃, CH₂CH₃, SO₂R, SO₂NR₂, or C(═O)NR₂;

or a pharmaceutically acceptable salt thereof.

In various embodiments, the invention provides a method of treatment of a patient for control of a condition wherein inhibition of KLF5 expression is medically indicated, comprising administering an effective amount of a compound of the invention to the patient. The method can be effective to delay or prevent colon cancer onset, halt growth of existing tumors, and/or decrease reoccurrence. For example, the condition can comprise colorectal cancer, diabetes, obesity, lipid homeostasis, cardiovascular disease, or arthritis.

### BRIEF DESCRIPTION OF THE FIGURES

FIG. 1A shows the chemical structure of compound 1, designated ML264.

FIG. 1B shows gel electrophoresis/autoradiographic evidence that treatment with a representative compound of the invention termed ML264 and also named compound 1. The chemical structure of this compound is described using Formula (I) in which R¹=(E)-2-(3-chlorophenyl)ethenyl, R²═H, R³=4-tetrahydro-2H-thiopyran 1,1-dioxide, R⁴=Me, X═NH, Y=absent). ML264 lowers KLF5 protein levels in multiple colon cancer cell lines. KLF5-lowering correlates with anti-proliferative activity, with less potent analogs of this compound, termed CID 5951923, CID46931037, and CID 46931043 having lesser effects on KLF5 expression than ML264.

FIG. 2 shows results of a luciferase assay of compound 1 (ML264) in the DLD-1/pGL4.18KLF5p cell line.

FIGS. 3A-F shows cell-based inhibition curves for ML264 in cell types DLD-1, HCT116, HT29, SW620, RKO, and IEC-6, respectively. ML264 halts DLD-1 viability (IC₅₀=29 nM) with high maximal effect (>90%). It has significant effects at submicromolar doses on other cell types as well, including HCT116, HT29, and SW620. The IEC-6 anti-target (lacking KLF5) is largely unaffected, with inhibition below 50% at the highest dose.

FIG. 4 shows the effect of ML264 on the cell cycle profile in the DLD-1 cell line.

FIGS. 5A and 5B show inhibition of human KLF5 promoter in DLD-1 stable cell line and inhibition of viability of DLD-1 cell line (compounds included: ML264, CIDs: 71449012 and 71449013. For compound structures, see Table 1.

### DETAILED DESCRIPTION

All patents and publications referred to herein are incorporated by reference herein to the same extent as if each individual publication was specifically and individually indicated to be incorporated by reference in its entirety.

Aspects of the present disclosure employ, unless otherwise indicated, techniques of chemistry, and the like, which are within the skill of the art. Such techniques are explained fully in the literature. Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As used in the specification and the appended claims, the singular forms “a,” “an” and “the” include plural referents unless the context clearly dictates otherwise.

The term “about” as used herein, when referring to a numerical value or range, allows for a degree of variability in the value or range, for example, within 10%, or within 5% of a stated value or of a stated limit of a range.

As used herein, “individual” (as in the subject of the treatment) or “patient” means both mammals and non-mammals. Mammals include, for example, humans; non-human primates, e.g. apes and monkeys; and non-primates, e.g. dogs, cats, cattle, horses, sheep, and goats. Non-mammals include, for example, fish and birds.

The term “disease” or “disorder” or “malcondition” are used interchangeably, and are used to refer to diseases or conditions wherein expression of KLF5 plays a role in the biochemical mechanisms involved in the disease or malcondition or symptom(s) thereof such that a therapeutically beneficial effect can be achieved by acting on the expression of KLF5, i.e., inhibiting the expression of KLF5.

The expression “effective amount”, when used to describe therapy to an individual suffering from a disorder, refers to the amount of a compound of the invention that is effective to inhibit or otherwise act on the expression of KLF5 in the individual's tissues wherein the expression of KLF5 involved in the disorder is active, wherein such inhibition or other action occurs to an extent sufficient to produce a beneficial therapeutic effect.

“Substantially” as the term is used herein means completely or almost completely; for example, a composition that is “substantially free” of a component either has none of the component or contains such a trace amount that any relevant functional property of the composition is unaffected by the presence of the trace amount, or a compound is “substantially pure” is there are only negligible traces of impurities present.

“Treating” or “treatment” within the meaning herein refers to an alleviation of symptoms associated with a disorder or disease, or inhibition of further progression or worsening of those symptoms, or prevention or prophylaxis of the disease or disorder, or curing the disease or disorder. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms, or prevents or provides prophylaxis for the disorder or condition. In particular, a “therapeutically effective amount” refers to an amount effective, at dosages and for periods of time necessary, to achieve the desired therapeutic result. A therapeutically effective amount is also one in which any toxic or detrimental effects of compounds of the invention are outweighed by the therapeutically beneficial effects.

Phrases such as “under conditions suitable to provide” or “under conditions sufficient to yield” or the like, in the context of methods of synthesis, as used herein refers to reaction conditions, such as time, temperature, solvent, reactant concentrations, and the like, that are within ordinary skill for an experimenter to vary, that provide a useful quantity or yield of a reaction product. It is not necessary that the desired reaction product be the only reaction product or that the starting materials be entirely consumed, provided the desired reaction product can be isolated or otherwise further used.

By “chemically feasible” is meant a bonding arrangement or a compound where the generally understood rules of organic structure are not violated; for example a structure within a definition of a claim that would contain in certain situations a pentavalent carbon atom that would not exist in nature would be understood to not be within the claim. The structures disclosed herein, in all of their embodiments are intended to include only “chemically feasible” structures, and any recited structures that are not chemically feasible, for example in a structure shown with variable atoms or groups, are not intended to be disclosed or claimed herein.

An “analog” of a chemical structure, as the term is used herein, refers to a chemical structure that preserves substantial similarity with the parent structure, although it may not be readily derived synthetically from the parent structure. A related chemical structure that is readily derived synthetically from a parent chemical structure is referred to as a “derivative.”

When a substituent is specified to be an atom or atoms of specified identity, “or a bond”, a configuration is referred to when the substituent is “a bond” that the groups that are immediately adjacent to the specified substituent are directly connected to each other in a chemically feasible bonding configuration.

All chiral, diastereomeric, racemic forms of a structure are intended, unless a particular stereochemistry or isomeric form is specifically indicated. In several instances though an individual stereoisomer is described among specifically claimed compounds, the stereochemical designation does not imply that alternate isomeric forms are less preferred, undesired, or not claimed. Compounds used in the present invention can include enriched or resolved optical isomers at any or all asymmetric atoms as are apparent from the depictions, at any degree of enrichment. Both racemic and diastereomeric mixtures, as well as the individual optical isomers can be isolated or synthesized so as to be substantially free of their enantiomeric or diastereomeric partners, and these are all within the scope of the invention.

As used herein, the terms “stable compound” and “stable structure” are meant to indicate a compound that is sufficiently robust to survive isolation to a useful degree of purity from a reaction mixture, and formulation into an efficacious therapeutic agent. Only stable compounds are contemplated herein.

A “small molecule” refers to an organic compound, including an organometallic compound, of a molecular weight less than about 2 kDa, that is not a polynucleotide, a polypeptide, a polysaccharide, or a synthetic polymer composed of a plurality of repeating units.

As to any of the groups described herein, which contain one or more substituents, it is understood that such groups do not contain any substitution or substitution patterns which are sterically impractical and/or synthetically non-feasible. In addition, the compounds of this disclosed subject matter include all stereochemical isomers arising from the substitution of these compounds.

When a group is recited, wherein the group can be present in more than a single orientation within a structure resulting in more than single molecular structure, e.g., a carboxamide group C(═O)NR, it is understood that the group can be present in any possible orientation, e.g., X—C(═O)N(R)—Y or X—N(R)C(═O)—Y, unless the context clearly limits the orientation of the group within the molecular structure.

When a group, e.g., an “alkyl” group, is referred to without any limitation on the number of atoms in the group, it is understood that the claim is definite and limited with respect the size of the alkyl group, both by definition; i.e., the size (the number of carbon atoms) possessed by a group such as an alkyl group is a finite number, bounded by the understanding of the person of ordinary skill as to the size of the group as being reasonable for a molecular entity; and by functionality, i.e., the size of the group such as the alkyl group is bounded by the functional properties the group bestows on a molecule containing the group such as solubility in aqueous or organic liquid media. Therefore, a claim reciting an “alkyl” or other chemical group or moiety is definite and bounded.

In general, “substituted” refers to an organic group as defined herein in which one or more bonds to a hydrogen atom contained therein are replaced by one or more bonds to a non-hydrogen atom such as, but not limited to, a halogen (i.e., F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxylamines, nitriles, nitro groups, N-oxides, hydrazides, azides, and enamines; and other heteroatoms in various other groups. Non-limiting examples of substituents J that can be bonded to a substituted carbon (or other) atom include F, Cl, Br, I, OR′, OC(O)N(R′)₂, CN, NO, NO₂, ONO₂, azido, CF₃, OCF₃, R′, O (oxo), S (thiono), methylenedioxy, ethylenedioxy, N(R′)₂, SR′, SOR′, SO₂R′, SO₂N(R′)₂, SO₃R′, C(O)R′, C(O)C(O)R′, C(O)CH₂C(O)R′, C(S)R′, C(O)OR′, OC(O)R′, C(O)N(R′)₂, OC(O)N(R′)₂, C(S)N(R′)₂, (CH₂)₀₋₂N(R′)C(O)R′, (CH₂)₀₋₂N(R′)N(R′)₂, N(R′)N(R′)C(O)R′, N(R′)N(R′)C(O)OR′, N(R′)N(R′)CON(R′)₂, N(R′)SO₂R′, N(R′)SO₂N(R′)₂, N(R′)C(O)OR′, N(R′)C(O)R′, N(R′)C(S)R′, N(R′)C(O)N(R′)₂, N(R′)C(S)N(R′)₂, N(COR′)COR′, N(OR′)R′, C(═NH)N(R′)₂, C(O)N(OR′)R′, or C(═NOR′)R′ wherein R′ can be hydrogen or a carbon-based moiety, and wherein the carbon-based moiety can itself be further substituted; for example, wherein R′ can be hydrogen, alkyl, acyl, cycloalkyl, aryl, aralkyl, heterocyclyl, heteroaryl, or heteroarylalkyl, wherein any alkyl, acyl, cycloalkyl, aryl, aralkyl, heterocyclyl, heteroaryl, or heteroarylalkyl or R′ can be independently mono- or multi-substituted with J; or wherein two R′ groups bonded to a nitrogen atom or to adjacent nitrogen atoms can together with the nitrogen atom or atoms form a heterocyclyl, which can be mono- or independently multi-substituted with J.

Substituted alkyl, alkenyl, alkynyl, cycloalkyl, and cycloalkenyl groups as well as other substituted groups also include groups in which one or more bonds to a hydrogen atom are replaced by one or more bonds, including double or triple bonds, to a carbon atom, or to a heteroatom such as, but not limited to, oxygen in carbonyl (oxo), carboxyl, ester, amide, imide, urethane, and urea groups; and nitrogen in imines, hydroxyimines, oximes, hydrazones, amidines, guanidines, and nitriles.

Substituted ring groups such as substituted cycloalkyl, aryl, heterocyclyl and heteroaryl groups also include rings and fused ring systems in which a bond to a hydrogen atom is replaced with a bond to a carbon atom. Therefore, substituted cycloalkyl, aryl, heterocyclyl and heteroaryl groups can also be substituted with alkyl, alkenyl, and alkynyl groups as defined herein.

By a “ring system” as the term is used herein is meant a moiety comprising one, two, three or more rings, which can be substituted with non-ring groups or with other ring systems, or both, which can be fully saturated, partially unsaturated, fully unsaturated, or aromatic, and when the ring system includes more than a single ring, the rings can be fused, bridging, or spirocyclic. By “spirocyclic” is meant the class of structures wherein two rings are fused at a single tetrahedral carbon atom, as is well known in the art.

A ring system can be monocyclic, bicyclic, tricyclic, or polycyclic, as is well known in the art. Any such ring system can be substituted, i.e., can bear one or more substituent groups as defined herein, bonded to an available position of the ring system. Visual structural representations such as

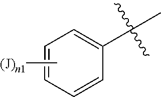

is intended to indicate a ring, bonded at the position indicated by the wavy line transecting the bond shown, and bearing n1 J groups at any available positions on the ring. For example, in the phenyl ring shown, there are five available positions for substitution, two ortho, two meta, and one para to the point of bonding, as is well known in the art. A visual structural representation such as

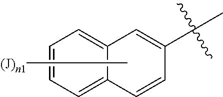

is intended to indicate a bicyclic ring system, bonded at the position indicated by the wavy line transecting the bond shown, and bearing n1 J groups at any available positions on each of the two rings. If it is intended that only one ring of a bicyclic (or higher cyclic) ring system can bear n1 J substituent groups, the system is depicted according to the following formula

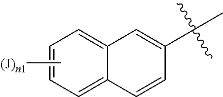

that is, with the line indicating the J groups only touching the single ring that is thus substituted.

Alkyl groups include straight chain and branched alkyl groups and cycloalkyl groups having from 1 to about 20 carbon atoms, and typically from 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed above, for example, amino, hydroxy, cyano, carboxy, nitro, thio, alkoxy, and halogen groups.

Cycloalkyl groups are cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, bicycloalkyl and tricycloalkyl groups such as norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined above. Representative substituted cycloalkyl groups can be mono-substituted or substituted more than once, such as, but not limited to, 2,2-, 2,3-, 2,4- 2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

The terms “carbocyclic,” “carbocyclyl,” and “carbocycle” denote a ring structure wherein the atoms of the ring are carbon, such as a cycloalkyl group or an aryl group. In some embodiments, the carbocycle has 3 to 8 ring members, whereas in other embodiments the number of ring carbon atoms is 4, 5, 6, or 7. Unless specifically indicated to the contrary, the carbocyclic ring can be substituted with as many as N-1 substituents wherein N is the size of the carbocyclic ring with, for example, alkyl, alkenyl, alkynyl, amino, aryl, hydroxy, cyano, carboxy, heteroaryl, heterocyclyl, nitro, thio, alkoxy, and halogen groups, or other groups as are listed above. A carbocyclyl ring can be a cycloalkyl ring, a cycloalkenyl ring, or an aryl ring. A carbocyclyl can be monocyclic or polycyclic, and if polycyclic each ring can be independently be a cycloalkyl ring, a cycloalkenyl ring, or an aryl ring.

(Cycloalkyl)alkyl groups, also denoted cycloalkylalkyl, are alkyl groups as defined above in which a hydrogen or carbon bond of the alkyl group is replaced with a bond to a cycloalkyl group as defined above.

Alkenyl groups include straight and branched chain and cyclic alkyl groups as defined above, except that at least one double bond exists between two carbon atoms. Thus, alkenyl groups have from 2 to about 20 carbon atoms, and typically from 2 to 12 carbons or, in some embodiments, from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

Aryl groups are cyclic aromatic hydrocarbons that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined above. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed above. Aryl groups can also bear fused rings, such as fused cycloalkyl rings, within the meaning herein. For example, a tetrahydronaphthyl ring is an example of an aryl group within the meaning herein. Accordingly, an aryl ring includes, for example, a partially hydrogenated system, which can be unsubstituted or substituted, and includes one or more aryl rings substituted with groups such as alkyl, alkoxyl, cycloalkyl, cycloalkoxyl, cycloalkylalkyl, cycloalkoxyalkyl, and the like, and also fused with, e.g., a cycloalkyl ring.

Aralkyl groups are alkyl groups as defined above in which a hydrogen or carbon bond of an alkyl group is replaced with a bond to an aryl group as defined above. Representative aralkyl groups include benzyl and phenylethyl groups and fused (cycloalkylaryl)alkyl groups such as 4-ethyl-indanyl. Aralkenyl group are alkenyl groups as defined above in which a hydrogen or carbon bond of an alkyl group is replaced with a bond to an aryl group as defined above.

An arylalkenyl group, as the term is used herein, refers to an alkenyl compound that is substituted with an aryl group; for example, a cinnamyl group is an arylalkenyl group, and a 3-phenylprop-1enyl group is an arylalkenyl group. The aryl and alkenyl unsaturations need not be conjugated.

Heterocyclyl groups or the term “heterocyclyl” includes aromatic and non-aromatic ring compounds containing 3 or more ring members, of which, one or more is a heteroatom such as, but not limited to, N, O, and S. The sulfur S can be in various oxidized forms, such as sulfide S, sulfoxide S(O) or sulfone S(O)₂. Thus a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. Heterocyclyl groups can be monocyclic, or polycyclic, such as bicyclic, tricyclic, or higher cyclic forms. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms sums up to equal the total number of ring atoms. A heterocyclyl ring can also include one or more double bonds. A heteroaryl ring is an embodiment of a heterocyclyl group. The phrase “heterocyclyl group” includes fused ring species including those comprising fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed above. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed above.

Heteroaryl groups are aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, thiadiazolyl, pyridinyl, pyrimidinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed above. Representative substituted heteroaryl groups can be substituted one or more times with groups such as those listed above.

A heteroarylalkenyl group is an alkenyl group substituted with a heteroaryl group; for example, a pyridylethenyl group is a heteroarylalkenyl group and a 3-(2-pyridyl)-prop-1-enyl group are each a heteroarylalkenyl group within the meaning herein. The heteroaryl and alkenyl unsaturations need not be conjugated.

Standard abbreviations for chemical groups such as are well known in the art can be used herein, and are within ordinary knowledge; e.g., Me=methyl, Et=ethyl, i-Pr=isopropyl, Bu=n-butyl, t-Bu=tert-butyl, Ph=phenyl, Bn=benzyl, Ac=acetyl, Bz=benzoyl, and the like.

A “salt” as is well known in the art includes an organic compound such as a carboxylic acid, a sulfonic acid, or an amine, in ionic form, in combination with a counterion. For example, acids in their anionic form can form salts with cations such as metal cations, for example sodium, potassium, and the like; with ammonium salts such as NH₄⁺ or the cations of various amines, including tetraalkyl ammonium salts such as tetramethylammonium, or other cations such as trimethylsulfonium, and the like. A “pharmaceutically acceptable” or “pharmacologically acceptable” salt is a salt formed from an ion that has been approved for human consumption and is generally non-toxic, among many examples are salts such a chloride salt or a sodium salt. A “zwitterion” is an internal salt such as can be formed in a molecule that has at least two ionizable groups, one forming an anion and the other a cation, which serve to balance each other. For example, amino acids such as glycine can exist in a zwitterionic form. A “zwitterion” is a salt within the meaning herein. The compounds of the present invention may take the form of salts. The term “salts” embraces addition salts of free acids or free bases which are compounds of the invention. Salts can be “pharmaceutically-acceptable salts.” The term “pharmaceutically-acceptable salt” refers to salts which possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds of the invention.

Suitable pharmaceutically-acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Examples of pharmaceutically unacceptable acid addition salts include, for example, perchlorates and tetrafluoroborates.

Suitable pharmaceutically acceptable base addition salts of compounds of the invention include, for example, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylenediamine, chloroprocaine, choline, diethanolamine, ethylenediamine, meglumine (N-methylglucamine) and procaine. Examples of pharmaceutically unacceptable base addition salts include lithium salts and cyanate salts. Although pharmaceutically unacceptable salts are not generally useful as medicaments, such salts may be useful, for example, as intermediates in the synthesis of Formula (I) compounds, for example in their purification by recrystallization. All of these salts may be prepared by conventional means from the corresponding compound according to Formula (I) by reacting, for example, the appropriate acid or base with the compound according to Formula (I). The term “pharmaceutically acceptable salts” refers to nontoxic inorganic or organic acid and/or base addition salts, see, for example, Lit et al., Salt Selection for Basic Drugs (1986), Int J. Pharm., 33, 201-217, incorporated by reference herein.

A “hydrate” is a compound that exists in a composition with water molecules. The composition can include water in stoichiometric quantities, such as a monohydrate or a dihydrate, or can include water in random amounts. As the term is used herein a “hydrate” refers to a solid form, i.e., a compound in water solution, while it may be hydrated, is not a hydrate as the term is used herein.

A “solvate” is a similar composition except that a solvent other that water replaces the water. For example, methanol or ethanol can form an “alcoholate”, which can again be stoichiometric or non-stoichiometric. As the term is used herein a “solvate” refers to a solid form, i.e., a compound in solution in a solvent, while it may be solvated, is not a solvate as the term is used herein.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described. Moreover, where features or aspects of the invention are described in terms of Markush groups, those skilled in the art will recognize that the invention is also thereby described in terms of any combination of individual members or subgroups of members of Markush groups. Thus, for example, if X is described as selected from the group consisting of bromine, chlorine, and iodine, and Y is described as selected from the group consisting of methyl, ethyl, and propyl, claims for X being bromine and Y being methyl are fully described.

If a value of a variable that is necessarily an integer, e.g., the number of carbon atoms in an alkyl group or the number of substituents on a ring, is described as a range, e.g., 0-4, what is meant is that the value can be any integer between 0 and 4 inclusive, i.e., 0, 1, 2, 3, or 4.

In various embodiments, the compound or set of compounds, such as are used in the inventive methods, can be any one of any of the combinations and/or sub-combinations of the above-listed embodiments.

In various embodiments, a compound as shown in any of the Examples, or among the exemplary compounds, is provided. Provisos may apply to any of the disclosed categories or embodiments wherein any one or more of the other above disclosed embodiments or species may be excluded from such categories or embodiments.

The present invention further embraces isolated compounds of the invention. The expression “isolated compound” refers to a preparation of a compound of the invention, or a mixture of compounds the invention, wherein the isolated compound has been separated from the reagents used, and/or byproducts formed, in the synthesis of the compound or compounds. “Isolated” does not mean that the preparation is technically pure (homogeneous), but it is sufficiently pure to compound in a form in which it can be used therapeutically. Preferably an “isolated compound” refers to a preparation of a compound of the invention or a mixture of compounds of the invention, which contains the named compound or mixture of compounds of the invention in an amount of at least 10 percent by weight of the total weight. Preferably the preparation contains the named compound or mixture of compounds in an amount of at least 50 percent by weight of the total weight; more preferably at least 80 percent by weight of the total weight; and most preferably at least 90 percent, at least 95 percent or at least 98 percent by weight of the total weight of the preparation.

The compounds of the invention and intermediates may be isolated from their reaction mixtures and purified by standard techniques such as filtration, liquid-liquid extraction, solid phase extraction, distillation, recrystallization or chromatography, including flash column chromatography, or HPLC.

### Isomerism and Tautomerism in Compounds of the Invention

### Tautomerism

Within the present invention it is to be understood that a compound of the formula (I) or a salt thereof may exhibit the phenomenon of tautomerism whereby two chemical compounds that are capable of facile interconversion by exchanging a hydrogen atom between two atoms, to either of which it forms a covalent bond. Since the tautomeric compounds exist in mobile equilibrium with each other they may be regarded as different isomeric forms of the same compound. It is to be understood that the formulae drawings within this specification can represent only one of the possible tautomeric forms. However, it is also to be understood that the invention encompasses any tautomeric form, and is not to be limited merely to any one tautomeric form utilized within the formulae drawings. The formulae drawings within this specification can represent only one of the possible tautomeric forms and it is to be understood that the specification encompasses all possible tautomeric forms of the compounds drawn not just those forms which it has been convenient to show graphically herein. For example, tautomerism may be exhibited by a pyrazolyl group bonded as indicated by the wavy line. While both substituents would be termed a 4-pyrazolyl group, it is evident that a different nitrogen atom bears the hydrogen atom in each structure.

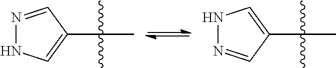 [SMILES: *c1cn[nH]c1 ; *c1cn[nH]c1]

Such tautomerism can also occur with substituted pyrazoles such as 3-methyl, 5-methyl, or 3,5-dimethylpyrazoles, and the like. Another example of tautomerism is amido-imido (lactam-lactim when cyclic) tautomerism, such as is seen in heterocyclic compounds bearing a ring oxygen atom adjacent to a ring nitrogen atom. For example, the equilibrium:

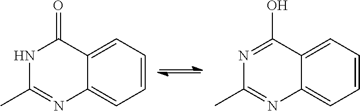

is an example of tautomerism. Accordingly, a structure depicted herein as one tautomer is intended to also include the other tautomer.

### Optical Isomerism

It will be understood that when compounds of the present invention contain one or more chiral centers, the compounds may exist in, and may be isolated as single and substantially pure enantiomeric or diastereomeric forms or as racemic mixtures. The present invention therefore includes any possible enantiomers, diastereomers, racemates or mixtures thereof of the compounds of the invention.

The isomers resulting from the presence of one chiral center comprise a pair of non-superimposable isomers that are called “enantiomers.” Single enantiomers of a pure compound are optically active, i.e., they are capable of rotating the plane of plane polarized light. Single enantiomers are designated according to the Cahn-Ingold-Prelog system. The priority of substituents is ranked based on atomic weights, a higher atomic weight, as determined by the systematic procedure, having a higher priority ranking. Once the priority ranking of the four groups is determined, the molecule is oriented so that the lowest ranking group is pointed away from the viewer. Then, if the descending rank order of the other groups proceeds clockwise, the molecule is designated as having an (R) absolute configuration, and if the descending rank of the other groups proceeds counterclockwise, the molecule is designated as having an (S) absolute configuration. In the example in the Scheme below, the Cahn-Ingold-Prelog ranking is A>B>C>D. The lowest ranking atom, D is oriented away from the viewer.

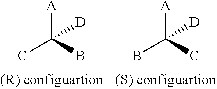

A carbon atom bearing the A-D atoms as shown above is known as a “chiral” carbon atom, and the position of such a carbon atom in a molecule is termed a “chiral center.” Compounds of the invention may contain more than one chiral center, and the configuration at each chiral center is described in the same fashion.

The present invention is meant to encompass diastereomers as well as their racemic and resolved, diastereomerically and enantiomerically pure forms and salts thereof. Diastereomeric pairs may be resolved by known separation techniques including normal and reverse phase chromatography, and crystallization.

“Isolated optical isomer” or “isolated enantiomer” means a compound which has been substantially purified from the corresponding optical isomer(s) of the same formula. Preferably, the isolated isomer is at least about 80%, more preferably at least 90% enantiomerically pure, even more preferably at least 98% enantiomerically pure, most preferably at least about 99% enantiomerically pure, by weight. By “enantiomeric purity” is meant the percent of the predominant enantiomer in an enantiomeric mixture of optical isomers of a compound. A pure single enantiomer has an enantiomeric purity of 100%.

Isolated optical isomers may be purified from racemic mixtures by well-known chiral separation techniques. According to one such method, a racemic mixture of a compound of the invention, or a chiral intermediate thereof, is separated into 99% wt. % pure optical isomers by HPLC using a suitable chiral column, such as a member of the series of DAICEL® CHIRALPAK® family of columns (Daicel Chemical Industries, Ltd., Tokyo, Japan). The column is operated according to the manufacturer's instructions.

Another well-known method of obtaining separate and substantially pure optical isomers is classic resolution, whereby a chiral racemic compound containing an ionized functional group, such as a protonated amine or carboxylate group, forms diastereomeric salts with an oppositely ionized chiral nonracemic additive. The resultant diastereomeric salt forms can then be separated by standard physical means, such as differential solubility, and then the chiral nonracemic additive may be either removed or exchanged with an alternate counter ion by standard chemical means, or alternatively the diastereomeric salt form may retained as a salt to be used as a therapeutic agent or as a precursor to a therapeutic agent.

### Rotational Isomerism

It is understood that due to chemical properties (i.e., resonance lending some double bond character to the C—N bond) of restricted rotation about the amide bond linkage (as illustrated below) it is possible to observe separate rotamer species and even, under some circumstances, to isolate such species (see below). It is further understood that certain structural elements, including steric bulk or substituents on the amide nitrogen, may enhance the stability of a rotamer to the extent that a compound may be isolated as, and exist indefinitely, as a single stable rotamer. The present invention therefore includes any possible stable rotamers of formula (I) which are biologically active in the treatment of cancer or other proliferative disease states.

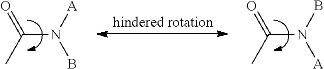

### Regioisomerism

The preferred compounds of the present invention have a particular spatial arrangement of substituents on the aromatic rings, which is related to the structure activity relationship demonstrated by the compound class. Often such substitution arrangement is denoted by a numbering system; however, numbering systems are often not consistent between different ring systems. In six-membered aromatic systems, the spatial arrangements are specified by the common nomenclature “para” for 1,4-substitution, “meta” for 1,3-substitution and “ortho” for 1,2-substitution as shown below.

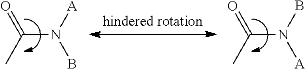

In various embodiments, the compound or set of compounds, such as are among the inventive compounds or are used in the inventive methods, can be any one of any of the combinations and/or sub-combinations of the above-listed embodiments.

### Isotope Substitution

It has become widely recognized by those skilled in the art that the incorporation of isotopic forms of an atom may impart useful properties. For example, a deuterium atom (²H) may be specifically introduced in place of a hydrogen atom which would otherwise represent the natural distribution of hydrogen isotopes, mostly ¹H. The use of one or more such isotopic substitutions may alter the properties of the resultant composition, including alterations in relevant properties in a treated animal, such as a longer half-life or duration of action of the composition. The isotope may also enable methods to detect the amount of the composition in affected tissue, such as by detection of radiation from isotopes such as ³H and ¹⁴C. Chemical methods for incorporating isotopes (examples including, but not limited to, ²H, ³H, ¹³C, ¹⁴C) are well-known in the art and the claims of this invention encompass such isotopic forms.

### Description

The invention is directed, in various embodiments, to small-molecule inhibitors of KLF5 expression, to methods of making the inhibitors, and to methods of treatment comprising administration of the inhibitors to patients suffering from an affliction wherein inhibition of KLF5 expression is medically indicated, including but not limited to colorectal cancer, diabetes, obesity, lipid homeostasis, cardiovascular disease, or arthritis.

The invention provides in various embodiments a KLF5 expression-inhibitory compound of formula (I)

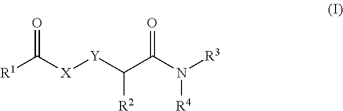

wherein

each R is independently H or (C₁-C₆)alkyl;

R¹ is aryl, arylalkenyl, heteroaryl, or heteroarylalkenyl, wherein any aryl or heteroaryl of R¹ is optionally mono- or independently multi-substituted with J;

X═CHR, O, or NR₂;

Y═CHR or absent;

R² is H, (C₁-C₆)alkyl, hydroxy(C₁-C₆)alkyl, (C₁-C₆)alkylNR₂, aryl, benzyl, p-hydroxybenzyl, (C₁-C₆)alkyl-C(═O)NR₂, (C₁-C₆)alkyl-C(═O)OR, or heteroaryl;

R³═H, (C₁-C₆)alkyl; (C₃-C₁₀)cycloalkyl, (C₁-C₆)alkylNR₂, (C₁-C₆)alkyl(C₃-C₁₀)cycloalkyl, or (C₁-C₆)alkyl-(3- to 10-membered heterocyclyl);

R⁴ is a 5-7 membered heterocyclyl comprising at least one C(═O)NR group or SO₂ group, optionally further comprising an additional NR group

J is halo, CF₃, OCF₃, CH₃, CH₂CH₃, SO₂R, SO₂NR₂, or C(═O)NR₂;

or a pharmaceutically acceptable salt thereof.

More specifically, for a compound of formula (I), R¹ can be a group of formula (II)

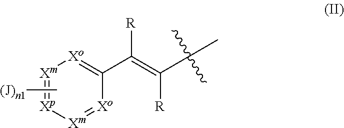

wherein

X^{o}, X^{m} and X^{p} are carbon or optionally any one or two independently selected X^{o}, X^{m}, or X^{p} is nitrogen;

R and J are as defined herein; and n1 is 0, 1, 2, 3, 4, or 5, provided that when any of X^{o}, X^{m}, or X^{p} are nitrogen, J is absent therefrom; and,

a wavy line indicates a point of bonding.

R¹ groups of this embodiment can be termed arylalkenyl and heteroarylalkenyl groups. For example, when all of X^{o}, X^{m}, and X^{p} are carbon bearing a respective hydrogen or J, and both R are hydrogen, the R¹ group is termed a cinnamyl group, which can be a substituted cinnamyl group when one or more J groups is present. When one or more R groups is non-hydrogen, all of X^{o}, X^{m}, and X^{p} are carbon bearing a respective hydrogen or J, the group can still be termed a substituted cinnamyl group, or can be referred to as a phenylalkenyl group, which can be substituted. When one or more of X^{o}, X^{m}, and X^{p} are non-carbon, e.g., nitrogen, the R¹ group can be termed a heteroarylalkenyl group.

In other embodiments according to the invention, R¹ can be of formula (III)

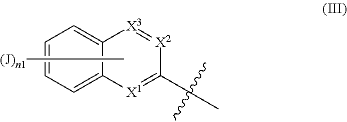

wherein

X¹, X² and X³ are each independently carbon or nitrogen;

the ring comprising X¹, X², and X³, or the aryl ring fused thereto, or both, can be substituted with n1 J group wherein J is halo, SO₂R, SO₂NR₂, or C(═O)NR₂ and n1 is 0, 1, 2, 3, 4, or 5; and,

a wavy line indicates a point of bonding.

More specifically, in compounds of this structural class, R¹ can be any one of

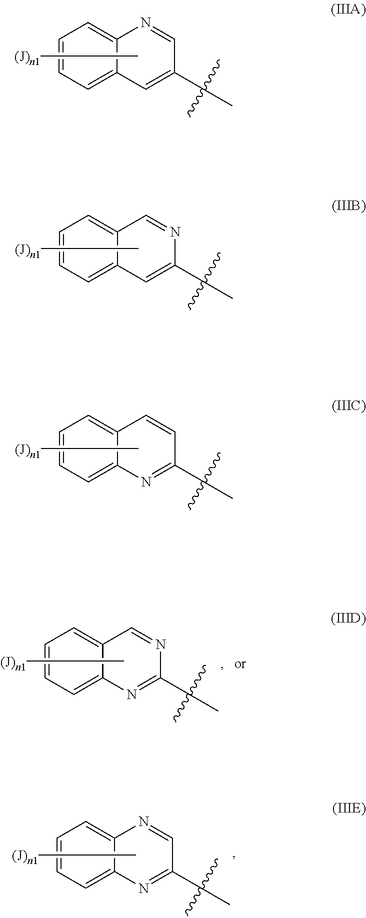

wherein J and n1 are as defined herein.

These compounds can be termed heteroaryl groups, such as quinolyl, isoquinolyl, quinazolyl, or quinoxalyl groups, any of which can be unsubstituted or can be mono- or independently multi-substituted with n1 J groups on either ring. The structural features shown above, with (J)n1 shown at the end of the line touching both rings of the bicyclic heteroaryl group indicates that each ring can bear up to n1 J groups, but limited by the number of available positions for substitution on each respective ring. For example, in formula (IIIE) above, it is apparent to the person of ordinary skill that the carbocyclic ring can bear up to four J groups, whereas the heterocyclic ring can only bear a single J group, based on determination of the number of positions on each respective ring available for substitution.

In other embodiments, R¹ can be of formula (IV)

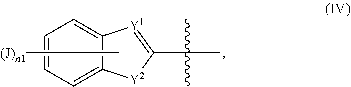

wherein

Y¹ is carbon or nitrogen, provided that when Y¹ is nitrogen, hydrogen and J are absent therefrom;

Y² is CR₂, NR, O, or S(O)q wherein q is 0, 1, or 2;

the ring comprising Y¹ and Y², or the aryl ring fused thereto, or both, can be substituted with n1 J group wherein J is halo, SO₂R, SO₂NR₂, or C(═O)NR₂ and n1 is 0, 1, 2, 3, 4, or 5.

For instance, in this structural class, R¹ can be any one of

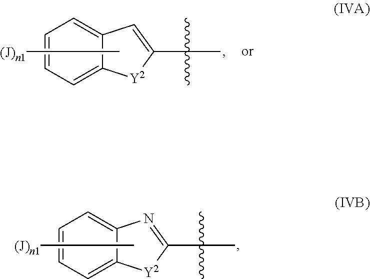

wherein Y², J, and n1 are as defined herein. Accordingly, among embodiments of compounds of formula (IVA), R¹ can include an indenyl (Y² is carbon), indolyl (Y² is nitrogen), benzofuranyl (Y² is oxygen) or benzothienyl (Y² is sulfur) group, any of which can be unsubstituted or can be mono- or independently multi-substituted with n1 J groups on either ring. For a compound of formula (IVB), R¹ can include a indolyl (Y² is carbon), benzimidazolyl (Y² is nitrogen), benzoxazolyl (Y² is oxygen), or benzthiazolyl (Y² is sulfur).

Compounds of the invention also include structures with variations in the groups bonding the R¹ group to the C(═O)N(R³)(R⁴) group, i.e., a moiety comprising —C(═O)—X—Y—CH(R²)—. In various embodiments, X is NR and Y is absent, providing an R¹-carbonyl amide of an amino acid amide, wherein R² is analogous to an amino acid sidechain. When Y is absent, the amino acid is an α-amino acid; when Y is CHR the amino acid is a β-amino acid. According, the R² group can be equivalent to a sidechain such as is found on any naturally occurring (e.g., ribosomal) amino acid, such as H, methyl, hydroxymethyl, carboxyalkyl, carboxamidoalkyl, benzyl, p-hydroxybenzyl, aminoalkyl, guanidinylalkyl, thioalkyl, methylthioalkyl, or similar group. The R² group can also be a group not found on any ribosomal amino acid, such as ethyl, hydroxyethyl, and the like.

In other embodiments, X is CHR, and Y can either be absent or be an independently selected CHR group, providing an R¹ ketone derivative of a carboxamide of N(R³)(R⁴).

The compound of formula (I) can bear as the R³ group bonded to the carboxamido nitrogen atom a hydrogen, (C₁-C₆)alkyl; (C₃-C₁₀)cycloalkyl, (C₁-C₆)alkylNR₂, (C₁-C₆)alkyl(C₃-C₁₀)cycloalkyl, or (C₁-C₆)alkyl-(3- to 10-membered heterocyclyl) group.

The compound of formula (I) can bear as the R⁴ group bonded to the carboxamido nitrogen atom a 5-7 membered heterocyclyl comprising at least one C(═O)NR group or SO₂ group, optionally further comprising an additional NR group.

For example, R⁴ can be any one of

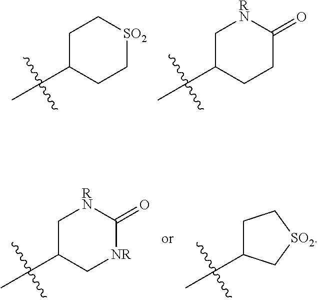

In specific embodiments, the compound can be any of the compounds shown in the Specific Compound Table, below. The table also shows the IC50 values (indicated by *), and % inhibition at 1 μM (indicated by **), determined as described below. As can be seen, compound 1, the most potent tested (designated as ML264), has an IC50 value around 80 nM, a potent inhibitor. Further investigative studies of ML264 (compound 1) are described in detail below.

TABLE 1
Specific Compound Table

| | | IC₅₀ (μM)* | |
|---|---|---|---|
| | | or % | |
| | | inhibition | |
| | | at 1 μM** | |
| | | or EC₅₀ cell | |
| Cpd | | viability | ESI-MS |
| # | Structure | (μM)*** | (m/z) |
| 1 | 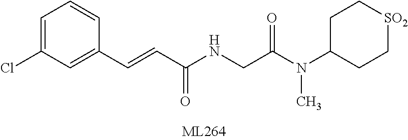 [SMILES: CN(C(=O)CNC(=O)/C=C/c1cccc(Cl)c1)C1CCS(=O)(=O)CC1] | 0.081* 0.054* .022*** | 385 (M + 1) |
| 2 | 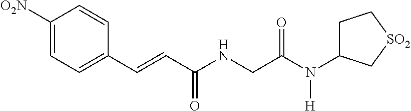 [SMILES: O=C(/C=C/c1ccc([N+](=O)[O-])cc1)NCC(=O)NC1CCS(=O)(=O)C1] | 25** | 368 |
| 3 | 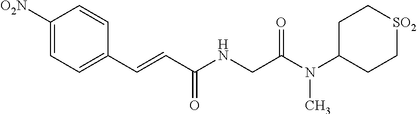 [SMILES: CN(C(=O)CNC(=O)/C=C/c1ccc([N+](=O)[O-])cc1)C1CCS(=O)(=O)CC1] | 25** | 396 |
| 4 | 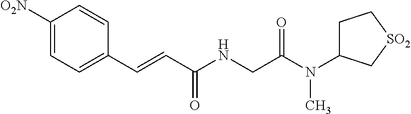 [SMILES: CN(C(=O)CNC(=O)/C=C/c1ccc([N+](=O)[O-])cc1)C1CCS(=O)(=O)C1] | 10** | 382 |
| 5 | 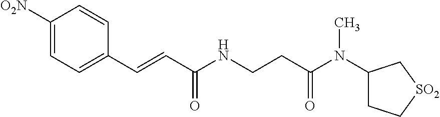 [SMILES: CN(C(=O)CCNC(=O)/C=C/c1ccc([N+](=O)[O-])cc1)C1CCS(=O)(=O)C1] | 15** | 396 |
| 6 | 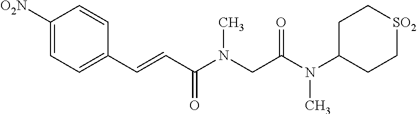 [SMILES: CN(CC(=O)N(C)C1CCS(=O)(=O)CC1)C(=O)/C=C/c1ccc([N+](=O)[O-])cc1] | 1** | 410 |
| 7 | 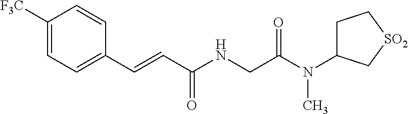 [SMILES: CN(C(=O)CNC(=O)/C=C/c1ccc(C(F)(F)F)cc1)C1CCS(=O)(=O)C1] | 9** | 405 |
| 8 | 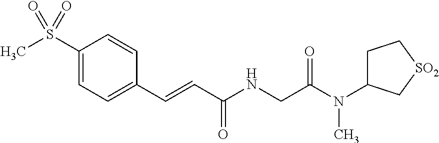 [SMILES: CN(C(=O)CNC(=O)/C=C/c1ccc(S(C)(=O)=O)cc1)C1CCS(=O)(=O)C1] | 26** | 415 |
| 9 | 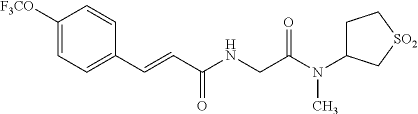 [SMILES: CN(C(=O)CNC(=O)/C=C/c1ccc(OC(F)(F)F)cc1)C1CCS(=O)(=O)C1] | 14** | 421 |
| 10 | 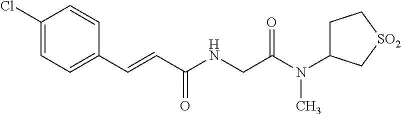 [SMILES: CN(C(=O)CNC(=O)/C=C/c1ccc(Cl)cc1)C1CCS(=O)(=O)C1] | 27** | 371 |
| 11 | 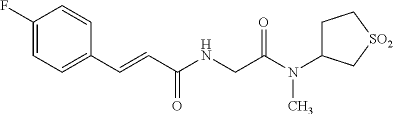 [SMILES: CN(C(=O)CNC(=O)/C=C/c1ccc(F)cc1)C1CCS(=O)(=O)C1] | 15** | 355 |
| 12 | 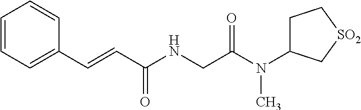 [SMILES: CN(C(=O)CNC(=O)/C=C/c1ccccc1)C1CCS(=O)(=O)C1] | 10** | 337 |
| 13 | 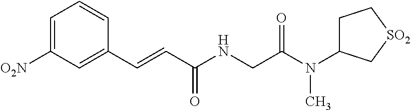 [SMILES: CN(C(=O)CNC(=O)/C=C/c1cccc([N+](=O)[O-])c1)C1CCS(=O)(=O)C1] | 60** 3.1* | 382 |
| 14 | 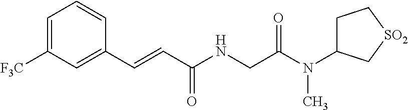 [SMILES: CN(C(=O)CNC(=O)/C=C/c1cccc(C(F)(F)F)c1)C1CCS(=O)(=O)C1] | 18** | 405 |
| 15 | 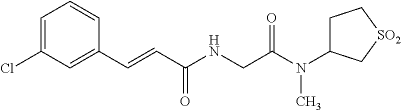 [SMILES: CN(C(=O)CNC(=O)/C=C/c1cccc(Cl)c1)C1CCS(=O)(=O)C1] | 46** 5.3* | 371 |
| 16 | 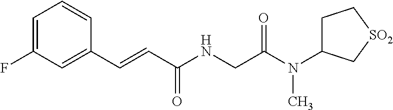 [SMILES: CN(C(=O)CNC(=O)/C=C/c1cccc(F)c1)C1CCS(=O)(=O)C1] | 31** | 355 |
| 17 | 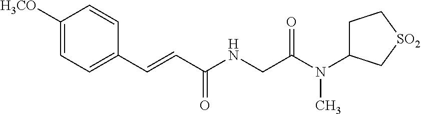 [SMILES: COc1ccc(/C=C/C(=O)NCC(=O)N(C)C2CCS(=O)(=O)C2)cc1] | 19** | 367 |
| 18 | 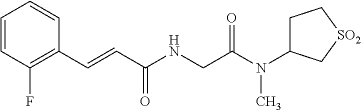 [SMILES: CN(C(=O)CNC(=O)/C=C/c1ccccc1F)C1CCS(=O)(=O)C1] | 31** | 355 |
| 19 | 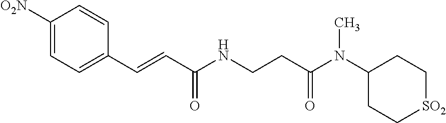 [SMILES: CN(C(=O)CCNC(=O)/C=C/c1ccc([N+](=O)[O-])cc1)C1CCS(=O)(=O)CC1] | 4** | 410 |
| 20 | 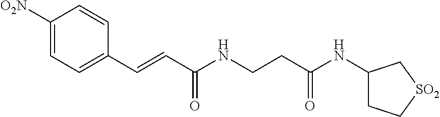 [SMILES: O=C(/C=C/c1ccc([N+](=O)[O-])cc1)NCCC(=O)NC1CCS(=O)(=O)C1] | 27** | 396 |
| 21 | 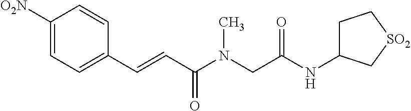 [SMILES: CN(CC(=O)NC1CCS(=O)(=O)C1)C(=O)/C=C/c1ccc([N+](=O)[O-])cc1] | −7** | 382 |
| 22 | 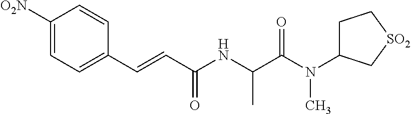 [SMILES: CC(NC(=O)/C=C/c1ccc([N+](=O)[O-])cc1)C(=O)N(C)C1CCS(=O)(=O)C1] | (R) 25** (S) 21** (R), (S) = Config. at methyl group | 396 |
| 23 | 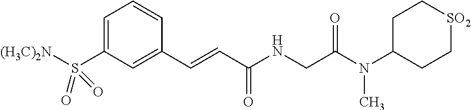 | 59* | 458 |
| 24 | 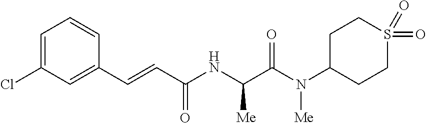 [SMILES: C[C@@H](NC(=O)/C=C/c1cccc(Cl)c1)C(=O)N(C)C1CCS(=O)(=O)CC1] | >100* | 399 |
| 25 | 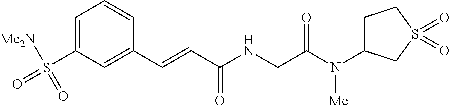 [SMILES: CN(C(=O)CNC(=O)/C=C/c1cccc(S(=O)(=O)N(C)C)c1)C1CCS(=O)(=O)C1] | >100* | 444 |
| 26 | 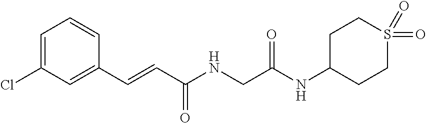 [SMILES: O=C(/C=C/c1cccc(Cl)c1)NCC(=O)NC1CCS(=O)(=O)CC1] | >100* | 371 |
| 27 | 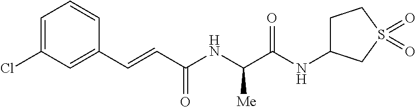 [SMILES: C[C@@H](NC(=O)/C=C/c1cccc(Cl)c1)C(=O)NC1CCS(=O)(=O)C1] | >100* | 371 |
| 28 | 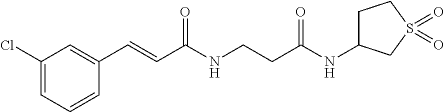 [SMILES: O=C(/C=C/c1cccc(Cl)c1)NCCC(=O)NC1CCS(=O)(=O)C1] | >100* | 371 |
| 29 | 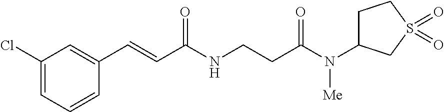 [SMILES: CN(C(=O)CCNC(=O)/C=C/c1cccc(Cl)c1)C1CCS(=O)(=O)C1] | >100* | 385 |
| 30 | 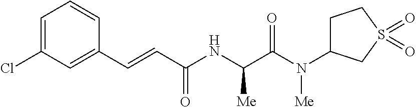 [SMILES: C[C@@H](NC(=O)/C=C/c1cccc(Cl)c1)C(=O)N(C)C1CCS(=O)(=O)C1] | >100* | 385 |
| 31 | 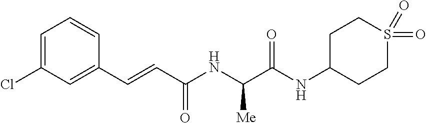 [SMILES: C[C@@H](NC(=O)/C=C/c1cccc(Cl)c1)C(=O)NC1CCS(=O)(=O)CC1] | >100* | 385 |
| 32 | 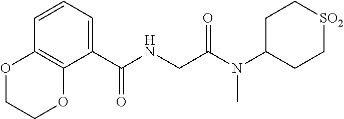 [SMILES: CN(C(=O)CNC(=O)c1cccc2c1OCCO2)C1CCS(=O)(=O)CC1] | >100* | 383 (M + 1) |
| 33 | 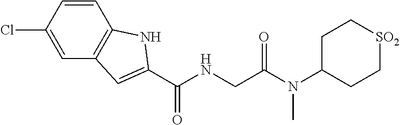 [SMILES: CN(C(=O)CNC(=O)c1cc2cc(Cl)ccc2[nH]1)C1CCS(=O)(=O)CC1] | >100* | 398 |
| 34 | 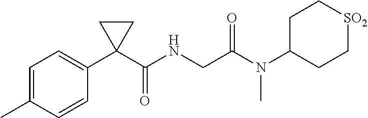 [SMILES: Cc1ccc(C2(C(=O)NCC(=O)N(C)C3CCS(=O)(=O)CC3)CC2)cc1] | >100* | 379 |
| 35 | 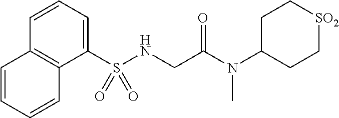 [SMILES: CN(C(=O)CNS(=O)(=O)c1cccc2ccccc12)C1CCS(=O)(=O)CC1] | >100* | 411 |
| 36 | 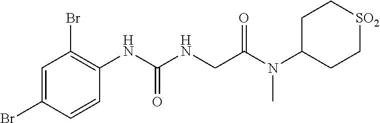 [SMILES: CN(C(=O)CNC(=O)Nc1ccc(Br)cc1Br)C1CCS(=O)(=O)CC1] | >100* | 498 |
| 37 | 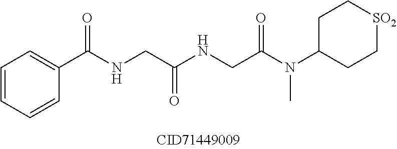 [SMILES: CN(C(=O)CNC(=O)CNC(=O)c1ccccc1)C1CCS(=O)(=O)CC1] | 20.49* .058*** | 382 |
| 38 | 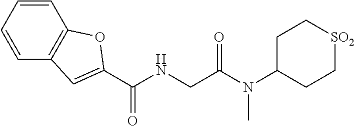 [SMILES: CN(C(=O)CNC(=O)c1cc2ccccc2o1)C1CCS(=O)(=O)CC1] | >100* | 365 |
| 39 | 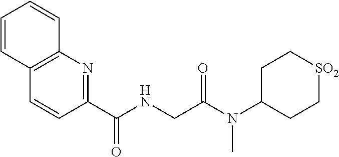 [SMILES: CN(C(=O)CNC(=O)c1ccc2ccccc2n1)C1CCS(=O)(=O)CC1] | >100* | 376 |
| 40 | 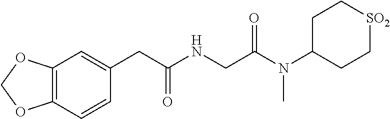 [SMILES: CN(C(=O)CNC(=O)Cc1ccc2c(c1)OCO2)C1CCS(=O)(=O)CC1] | >100* | 383 |
| 41 | 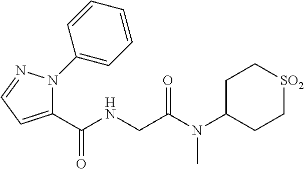 [SMILES: CN(C(=O)CNC(=O)c1ccnn1-c1ccccc1)C1CCS(=O)(=O)CC1] | >100* | 391 |
| 42 | 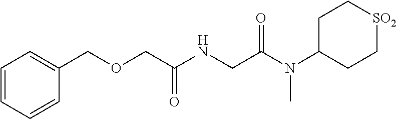 [SMILES: CN(C(=O)CNC(=O)COCc1ccccc1)C1CCS(=O)(=O)CC1] | >100* | 369 |
| 43 | 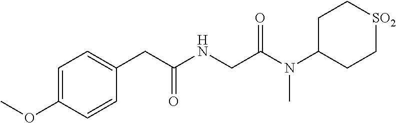 [SMILES: COc1ccc(CC(=O)NCC(=O)N(C)C2CCS(=O)(=O)CC2)cc1] | >100* | 369 |
| 44 | 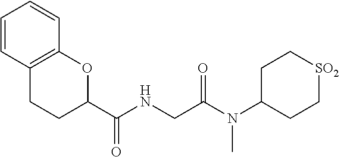 [SMILES: CN(C(=O)CNC(=O)C1CCc2ccccc2O1)C1CCS(=O)(=O)CC1] | >100* | 381 |
| 45 | 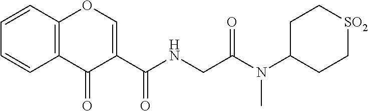 [SMILES: CN(C(=O)CNC(=O)c1coc2ccccc2c1=O)C1CCS(=O)(=O)CC1] | >100* | 393 |
| 46 | 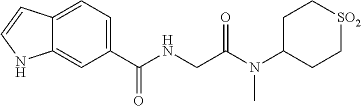 [SMILES: CN(C(=O)CNC(=O)c1ccc2cc[nH]c2c1)C1CCS(=O)(=O)CC1] | >100* | 364 |
| 47 | 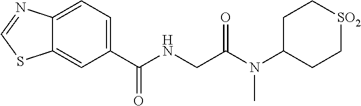 [SMILES: CN(C(=O)CNC(=O)c1ccc2ncsc2c1)C1CCS(=O)(=O)CC1] | >100* | 382 |
| 48 | 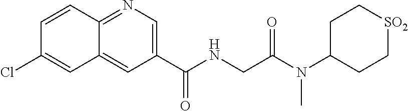 [SMILES: CN(C(=O)CNC(=O)c1cnc2ccc(Cl)cc2c1)C1CCS(=O)(=O)CC1] | >100* | 412 |
| 49 | 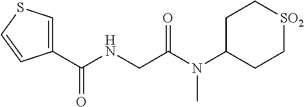 [SMILES: CN(C(=O)CNC(=O)c1ccsc1)C1CCS(=O)(=O)CC1] | >100* | 331 |
| 50 | 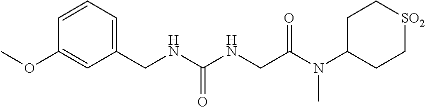 [SMILES: COc1cccc(CNC(=O)NCC(=O)N(C)C2CCS(=O)(=O)CC2)c1] | >100* | 384 |
| 51 | 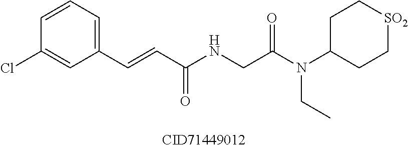 [SMILES: CCN(C(=O)CNC(=O)/C=C/c1cccc(Cl)c1)C1CCS(=O)(=O)CC1] | 0.072* 0.127*** | 399 |
| 52 | 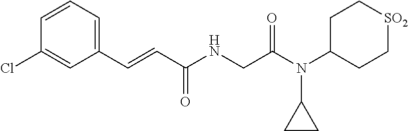 [SMILES: O=C(/C=C/c1cccc(Cl)c1)NCC(=O)N(C1CC1)C1CCS(=O)(=O)CC1] | >100* | 413 |
| 53C | 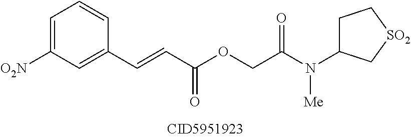 [SMILES: CN(C(=O)COC(=O)/C=C/c1cccc([N+](=O)[O-])c1)C1CCS(=O)(=O)C1] | 0.27* 1.51*** | |
| 54 | 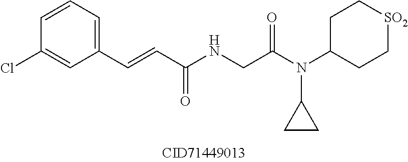 [SMILES: O=C(/C=C/c1cccc(Cl)c1)NCC(=O)N(C1CC1)C1CCS(=O)(=O)CC1] | 7.80* 0.39*** | |

DlD-1 4.9 cell line, measuring luciferase activity; IC50 (μM)* or % inhibition at 1 μM** or IC50 cell viability (μM)***.
C = comparative compound

In various embodiments, the invention provides a pharmaceutical composition comprising a compound of the invention, and a pharmaceutically acceptable excipient.

Another aspect of an embodiment of the invention provides compositions of the compounds of the invention, alone or in combination with another medicament. As set forth herein, compounds of the invention include stereoisomers, tautomers, solvates, prodrugs, pharmaceutically acceptable salts and mixtures thereof. Compositions containing a compound of the invention can be prepared by conventional techniques, e.g. as described in Remington: The Science and Practice of Pharmacy, 19th Ed., 1995, or later versions thereof, incorporated by reference herein. The compositions can appear in conventional forms, for example capsules, tablets, aerosols, solutions, suspensions or topical applications.

Typical compositions include a compound of the invention and a pharmaceutically acceptable excipient which can be a carrier or a diluent. For example, the active compound will usually be mixed with a carrier, or diluted by a carrier, or enclosed within a carrier which can be in the form of an ampoule, capsule, sachet, paper, or other container. When the active compound is mixed with a carrier, or when the carrier serves as a diluent, it can be solid, semi-solid, or liquid material that acts as a vehicle, excipient, or medium for the active compound. The active compound can be adsorbed on a granular solid carrier, for example contained in a sachet. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent can include any sustained release material known in the art, such as glyceryl monostearate or glyceryl distearate, alone or mixed with a wax.

The formulations can be mixed with auxiliary agents which do not deleteriously react with the active compounds. Such additives can include wetting agents, emulsifying and suspending agents, salt for influencing osmotic pressure, buffers and/or coloring substances preserving agents, sweetening agents or flavoring agents. The compositions can also be sterilized if desired.

The route of administration can be any route which effectively transports the active compound of the invention to the appropriate or desired site of action, such as oral, nasal, pulmonary, buccal, subdermal, intradermal, transdermal or parenteral, e.g., rectal, depot, subcutaneous, intravenous, intraurethral, intramuscular, intranasal, ophthalmic solution or an ointment, the oral route being preferred.

If a solid carrier is used for oral administration, the preparation can be tableted, placed in a hard gelatin capsule in powder or pellet form or it can be in the form of a troche or lozenge. If a liquid carrier is used, the preparation can be in the form of a syrup, emulsion, soft gelatin capsule or sterile injectable liquid such as an aqueous or non-aqueous liquid suspension or solution.

Injectable dosage forms generally include aqueous suspensions or oil suspensions which can be prepared using a suitable dispersant or wetting agent and a suspending agent Injectable forms can be in solution phase or in the form of a suspension, which is prepared with a solvent or diluent. Acceptable solvents or vehicles include sterilized water, Ringer's solution, or an isotonic aqueous saline solution. Alternatively, sterile oils can be employed as solvents or suspending agents. Preferably, the oil or fatty acid is non-volatile, including natural or synthetic oils, fatty acids, mono-, di- or tri-glycerides.

For injection, the formulation can also be a powder suitable for reconstitution with an appropriate solution as described above. Examples of these include, but are not limited to, freeze dried, rotary dried or spray dried powders, amorphous powders, granules, precipitates, or particulates. For injection, the formulations can optionally contain stabilizers, pH modifiers, surfactants, bioavailability modifiers and combinations of these. The compounds can be formulated for parenteral administration by injection such as by bolus injection or continuous infusion. A unit dosage form for injection can be in ampoules or in multi-dose containers.

The formulations of the invention can be designed to provide quick, sustained, or delayed release of the active ingredient after administration to the patient by employing procedures well known in the art. Thus, the formulations can also be formulated for controlled release or for slow release.

Compositions contemplated by the present invention can include, for example, micelles or liposomes, or some other encapsulated form, or can be administered in an extended release form to provide a prolonged storage and/or delivery effect. Therefore, the formulations can be compressed into pellets or cylinders and implanted intramuscularly or subcutaneously as depot injections. Such implants can employ known inert materials such as silicones and biodegradable polymers, e.g., polylactide-polyglycolide. Examples of other biodegradable polymers include poly(orthoesters) and poly(anhydrides).

For nasal administration, the preparation can contain a compound of the invention, dissolved or suspended in a liquid carrier, preferably an aqueous carrier, for aerosol application. The carrier can contain additives such as solubilizing agents, e.g., propylene glycol, surfactants, absorption enhancers such as lecithin (phosphatidylcholine) or cyclodextrin, or preservatives such as parabens.

For parenteral application, particularly suitable are injectable solutions or suspensions, preferably aqueous solutions with the active compound dissolved in polyhydroxylated castor oil.

Tablets, dragees, or capsules having talc and/or a carbohydrate carrier or binder or the like are particularly suitable for oral application. Preferable carriers for tablets, dragees, or capsules include lactose, corn starch, and/or potato starch. A syrup or elixir can be used in cases where a sweetened vehicle can be employed.

A typical tablet that can be prepared by conventional tableting techniques can contain:

Core:

| | |
|---|---|
| Active compound (as free compound or salt thereof) | 250 mg |
| Colloidal silicon dioxide (Aerosil ®) | 1.5 mg |
| Cellulose, microcryst. (Avicel ®) | 70 mg |
| Modified cellulose gum (Ac-Di-Sol ®) | 7.5 mg |
| Magnesium stearate | Ad. |

Coating:

| | |
|---|---|
| HPMC approx. | 9 mg |
| *Mywacett 9-40 T approx. | 0.9 mg |

*Acylated monoglyceride used as plasticizer for film coating.

A typical capsule for oral administration contains compounds of the invention (250 mg), lactose (75 mg) and magnesium stearate (15 mg). The mixture is passed through a 60 mesh sieve and packed into a No. 1 gelatin capsule. A typical injectable preparation is produced by aseptically placing 250 mg of compounds of the invention into a vial, aseptically freeze-drying and sealing. For use, the contents of the vial are mixed with 2 mL of sterile physiological saline, to produce an injectable preparation.

In various embodiments, the invention provides a method of treatment of a patient for control of a condition wherein inhibition of KLF5 expression is medically indicated, comprising administering an effective amount of a compound of claim 1 to the patient. For example, administration of a compound of the invention can be used to delay or prevent colon cancer onset, halt growth of existing tumors, and/or decrease reoccurrence. The medical condition can comprise colorectal cancer, diabetes, obesity, lipid homeostasis, cardiovascular disease, or arthritis, or any combination thereof.

The compounds of the invention can be administered to a mammal, especially a human in need of such treatment, prevention, elimination, alleviation or amelioration of a malcondition. Such mammals include also animals, both domestic animals, e.g. household pets, farm animals, and non-domestic animals such as wildlife.

The compounds of the invention are effective over a wide dosage range. For example, in the treatment of adult humans, dosages from about 0.05 to about 5000 mg, preferably from about 1 to about 2000 mg, and more preferably between about 2 and about 2000 mg per day can be used. A typical dosage is about 10 mg to about 1000 mg per day. In choosing a regimen for patients it can frequently be necessary to begin with a higher dosage and when the condition is under control to reduce the dosage. The exact dosage will depend upon the activity of the compound, mode of administration, on the therapy desired, form in which administered, the subject to be treated and the body weight of the subject to be treated, and the preference and experience of the physician or veterinarian in charge.

Generally, the compounds of the invention are dispensed in unit dosage form including from about 0.05 mg to about 1000 mg of active ingredient together with a pharmaceutically acceptable carrier per unit dosage.

Usually, dosage forms suitable for oral, nasal, pulmonal or transdermal administration include from about 125 μg to about 1250 mg, preferably from about 250 μg to about 500 mg, and more preferably from about 2.5 mg to about 250 mg, of the compounds admixed with a pharmaceutically acceptable carrier or diluent.

Dosage forms can be administered daily, or more than once a day, such as twice or thrice daily. Alternatively dosage forms can be administered less frequently than daily, such as every other day, or weekly, if found to be advisable by a prescribing physician.

The present invention is directed, in various embodiments, to small molecule inhibitors of Krüppel-like factor 5 (KLF5) expression, to methods of using the inhibitors, and to methods of making the inhibitors. In various embodiments, the invention provides a small molecule inhibitor of KLF5 expression which can be effective delay or prevent colon cancer onset, halt growth of existing tumors, and/or decrease reoccurrence. The compound can be effective vs many tumor types whose progression is in part mediated by KLF5, including colorectal cancers. Lowering KLF5 levels with an inhibitor of this invention may also impact other disease states, including diabetes, obesity, lipid homeostasis, cardiovascular disease, and arthritis. The inventors here have used an ultra-high throughput screening (uHTS) approach, follow-up mechanistic studies, and medicinal chemistry efforts aimed to enhance potency, selectivity, lead-like and drug-like properties¹⁶⁻²⁰ to discover potent inhibitors of KLF5 expression.

Compound 1, Designated ML264

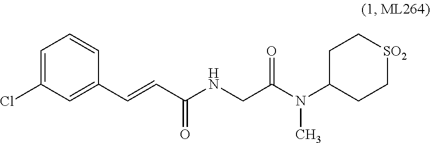 [SMILES: CN(C(=O)CNC(=O)/C=C/c1cccc(Cl)c1)C1CCS(=O)(=O)CC1]

was found to cytotoxic in many tumor cell types (Table 2, below). Moreover, cytotoxicity in general trends with KLF5 levels in the tumor types. As indicated in Table 2, basal levels of KLF5 mRNA expression for NCI60 cell lines are known and were presented in a publication.²¹ ML264 is found to be highly cytotoxic toward seven of the ten cell lines having the highest basal levels of KLF5 mRNA, including five colon cancer cell lines. Only the colon cancer cell line KM12, the ovarian cancer line IGROV1, and the non-small cell lung cancer line NCI-H322M are the NCI60 cell lines high with respect to KLF5 mRNA levels that are ML264-insensitive.

TABLE 2
Potency of ML-264 versus Tumor Cell Lines

| | IC50 | |
|---|---|---|
| Cell Line | (nM) | mRNA/protein levels |
| DLD1 (colon) | 29 | elevated KLF5 mRNA level |
| HT29 (colon) | 130 | elevated KLF5 mRNA level |
| A498 (renal) | 420 | |
| SW-620 (colon) | 430 | elevated KLF5 mRNA level |
| DU-145 (prostate) | 470 | |
| MDA-MB-231/ATCC | 530 | |
| (breast) | | |
| HCT-116 (colon) | 560 | KLF5 mRNA levels low but protein |
| | | level high by Western |
| HCC02998 (colon) | 660 | elevated KLF5 mRNA level |
| LOXIMVI (melanoma) | 710 | |
| UACC-62 (melanoma) | 980 | |
| MCF-7 (breast) | 1000 | |
| NCI-H226 (NSLCC) | 1000 | |
| COLO205 (colon) | 1100 | elevated KLF5 mRNA level |
| NCI-H522 (NSLCC) | 1200 | elevated KLF5 mRNA level |
| OVCAR-3 (ovarian) | 1200 | elevated KLF5 mRNA level |

The mRNA levels may not be truly reflective of cellular protein load, however. As an example, the ML264-sensitive colon cancer cell line HCT-116 does not show elevated basal levels of KLF5 mRNA but has elevated basal levels of KLF5 protein by Western blot. It is also possible that even cell types with normally low levels of KLF5 may be significantly affected by driving KLF5 levels still lower. Breast cancer cell lines, for example, have uniformly low KLF5 mRNA expression levels, and in fact a tumor-suppressor role of KLF5 in breast cancer has been proposed.²² The finding that breast cancer patients with higher KLF5 expression have shorter disease-free survival and reduced overall survival than patients with lower KLF5 expression¹³ seems to contradict this notion, suggesting that even low levels of KLF5 can drive progression of particularly aggressive tumors that normally (i.e., in genetically related but less aggressive tumors) have even lower KLF5 levels.

We have also looked for off-target activity of ML264, and it is inactive vs. 47 kinases that could give cell-based assay results falsely consistent with the intended mode of action. It also showed no significant inhibition (<<50%) at 10 μM vs. 67 protein targets of therapeutic and/or toxicological interest (GPCRs, ion channels, enzymes, etc.).

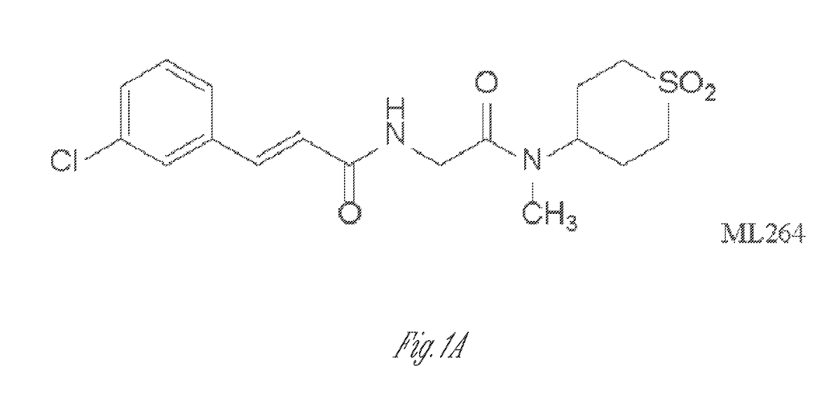

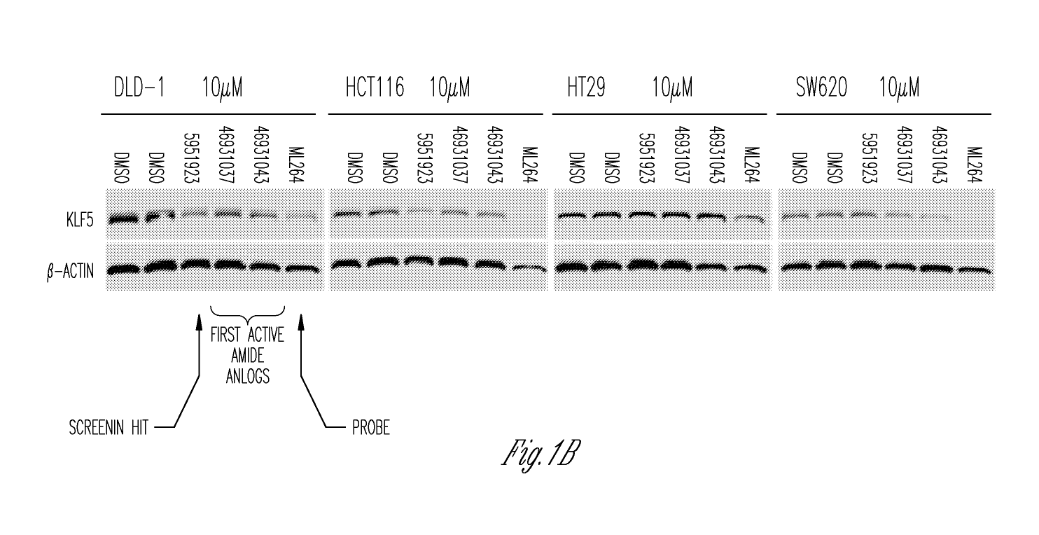

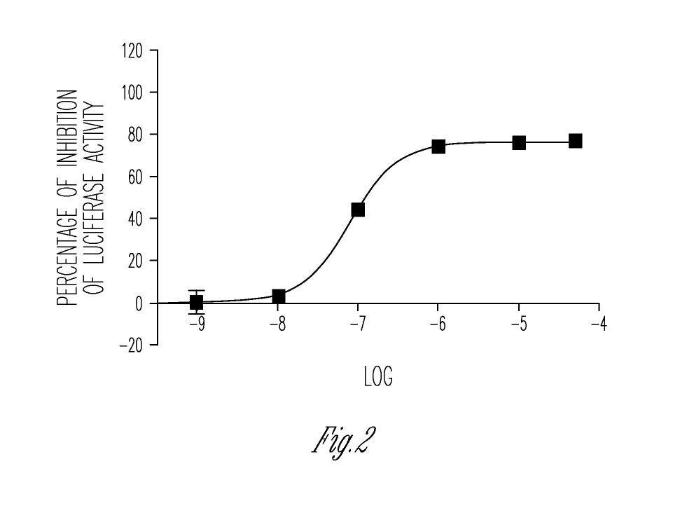

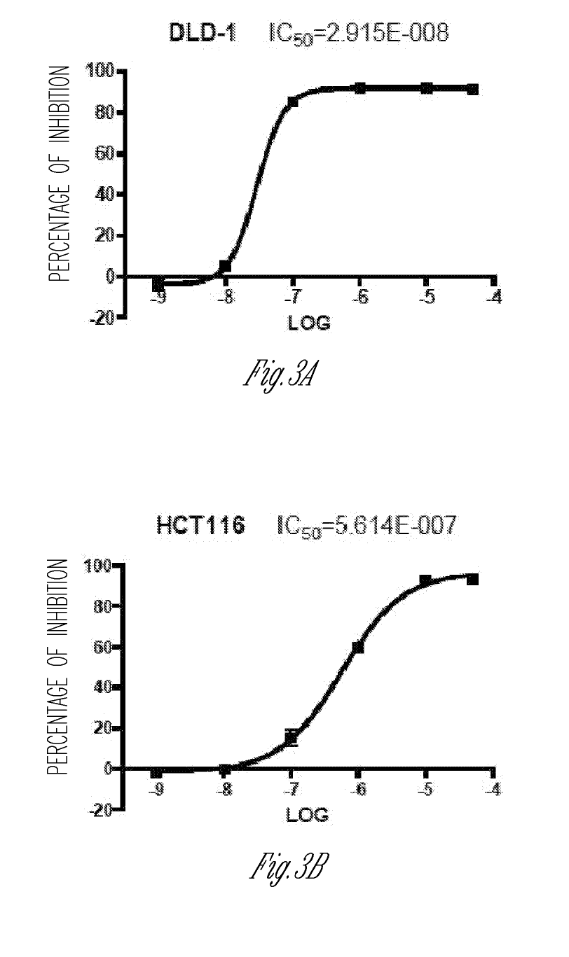

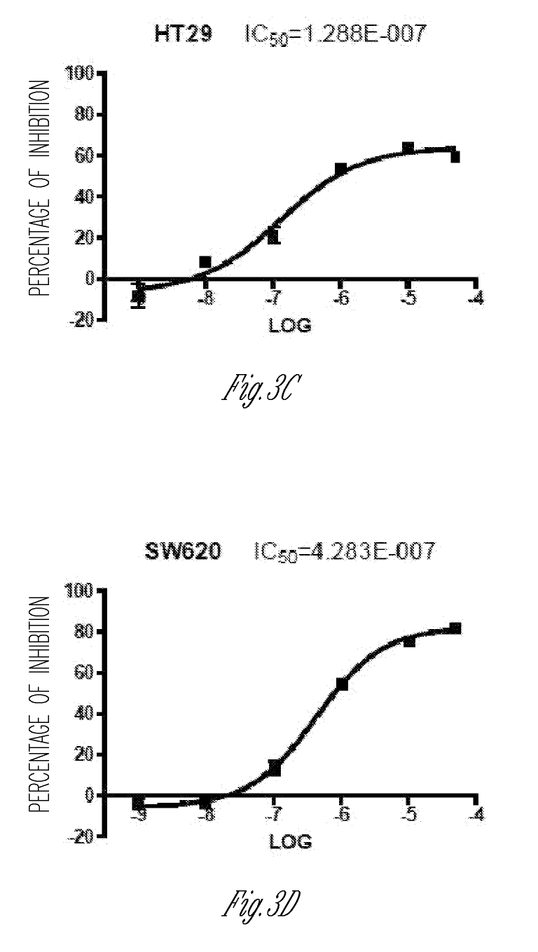

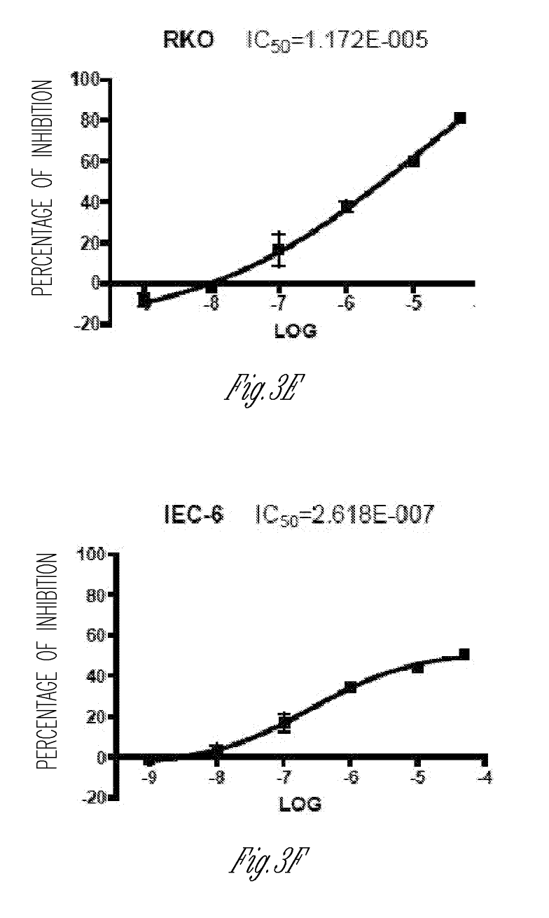

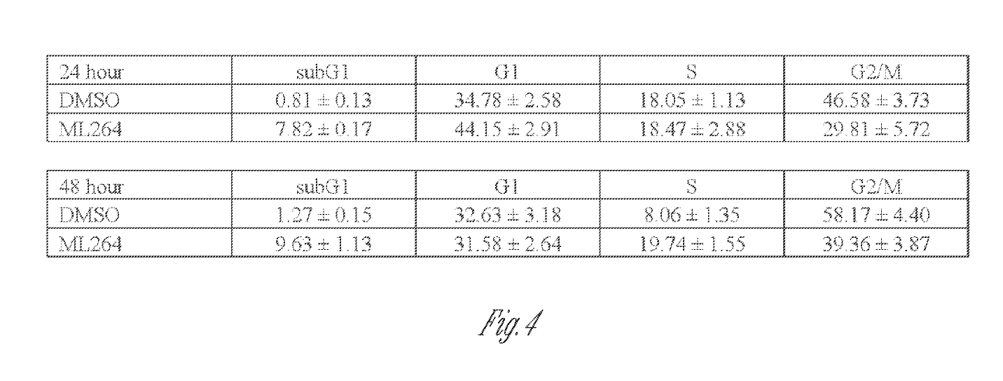

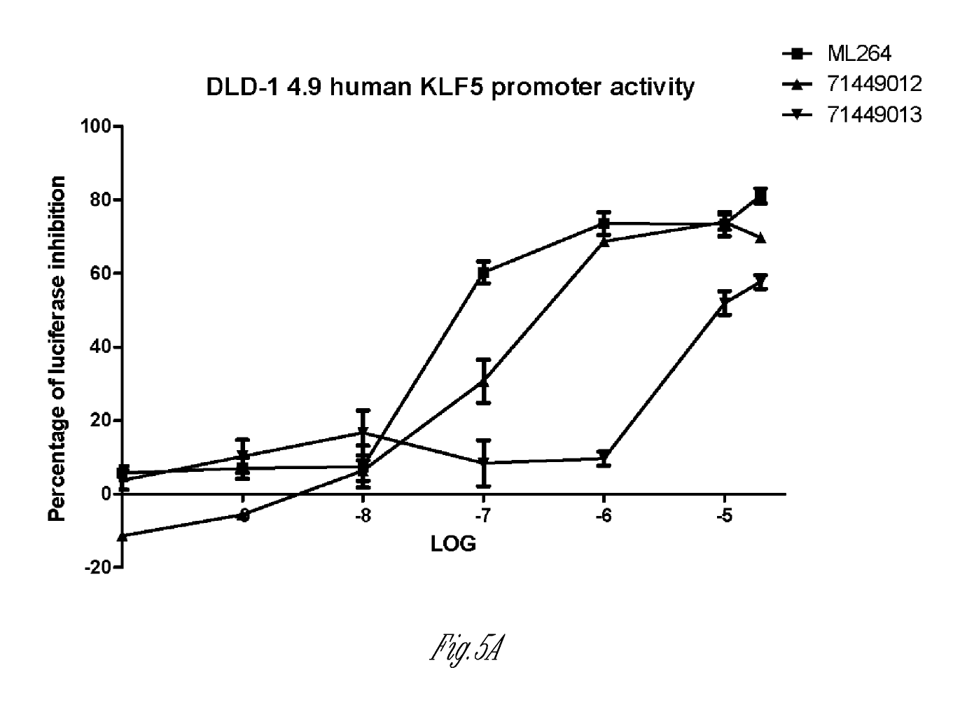

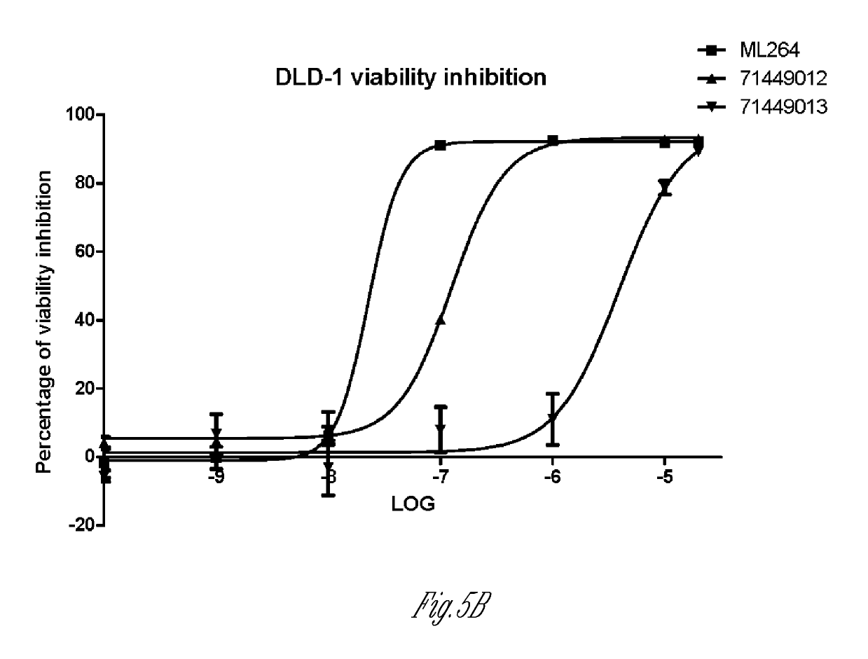

We have studied the effects of ML264 and analogs upon lowering KLF5 levels in colon cancer cell lines. ML264 significantly reduces KLF5 protein levels by Western blot (FIG. 1).

Our approach to a novel therapy targets KLF5 production rather than alternative strategies such as targeting its ubiquitin-mediated degradation or blocking interactions of KLF5 with other proteins. No prior art KLF5 inhibitors are to our knowledge highly effective in reducing KLF5 expression with clearly defined and suitably high mechanistic specificity.

### Synthesis of Compounds of the Invention

ML264 and most compounds of formula (I) are readily prepared in high yield in a few steps. The main synthesis route is shown in Synthetic Scheme 1. As an illustrative example, the synthesis of ML264 by this route is also shown. We have also developed an alternative synthesis route, shown in Synthetic Scheme 2 as applied to ML264.

### Synthetic Scheme 1 (General, and as Applied to ML264)

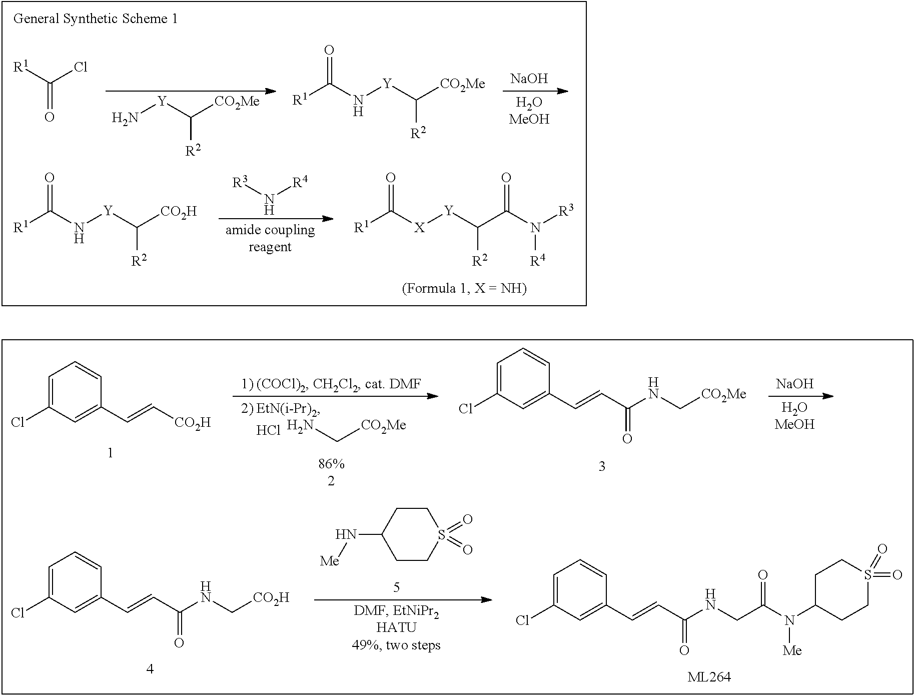

### Synthetic Scheme 2 (General, and as Applied to ML264)

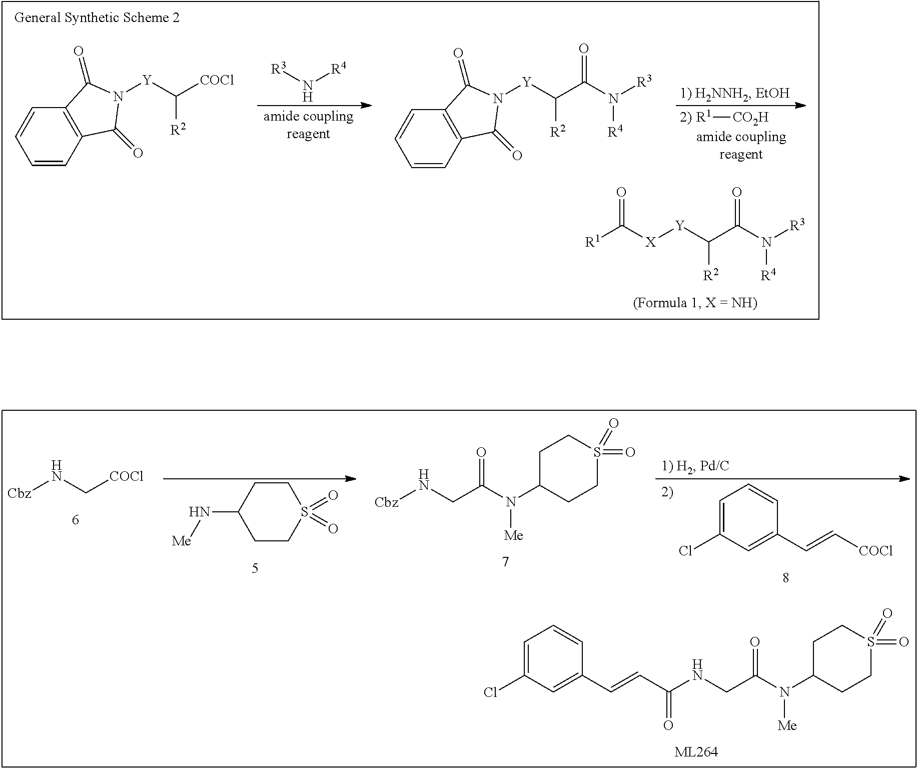

### Drug-Like Properties of Compounds of the Invention

Various chemical descriptors for ML264 were calculated using the Accelrys Pipeline Pilot software and by applying standard “rule-of-five” and other lead-likeness or drug-likeness criteria.¹⁶⁻²⁰ As shown in Table 3, below, ML264's structure prompts no significant concerns from a reactivity, stability, lead-likeness, drug-likeness, or general toxicity alert perspective.

TABLE 3
Drug-Like Properties of Compound 1

| | | Physical |
|---|---|---|
| Drug-like properties of ML264 | | Properties Measured at Scripps |
| Mol. Wt: 384.9 | H acceptors: 4 | Solubility in PBS = 18.7 μM |
| cLogP: 0.28 | H donors: 1 | Solubility in assay buffer = 35 μM |
| Log D_{7.4}: 1.00 | atom count: 25 | Stability (t_{1/2}) in PBS: >48 h |
| tPSA = 83.6 | rotatable bonds: 5 | Michael acceptor: No |
| | rings: 2 | (unchanged by LCMS after |
| | | incubation with 100 μM GSH) |

no stereocenters
no formal charge
no obvious toxicophores

### Examples

Synthesis of compounds of Formula 1 and synthetic intermediates are described. Variations of these standard methods such as known to those skilled in the art may also be used for their preparation.

### Example 1

### Preparation of ML264

### Step 1: (E)-methyl 2-(3-(3-chlorophenyl)acrylamido)acetate

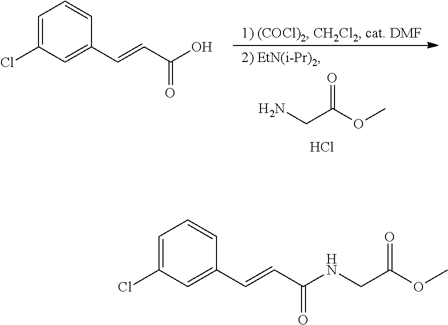

To (E)-3-(3-chlorophenyl)acrylic acid (1.0 g, 5.48 mmol) in dichloromethane (10 mL) was added anhydrous dimethylformamide (DMF) (0.08 mL, 1.0 mmol). This solution was cooled to 0° C. and then oxalyl dichloride (0.57 mL, 6.53 mmol) was added dropwise. The reaction was allowed to warm to room temperature. After two hours at room temperature, the reaction mixture was re-cooled to 0° C. and a mixture of the HCl salt of glycine methyl ester (1.38 g, 10.99 mmol) and diisopropyl ethyl amine (DIEA, 3.8 mL, 21.92 mmol) in dichloromethane (10 mL) was added slowly. The reaction was stirred at room temperature overnight. The solvent was removed in vacuo to obtain the crude product, which was purified by flash chromatography (AcOEt/Hex 10˜100%) to obtain the title compound, 1.19 g (86%). ESI-MS (m/z): 254, [M+1]⁺.

### Step 2. (E)-2-(3-(3-chlorophenyl)acrylamido)acetic acid

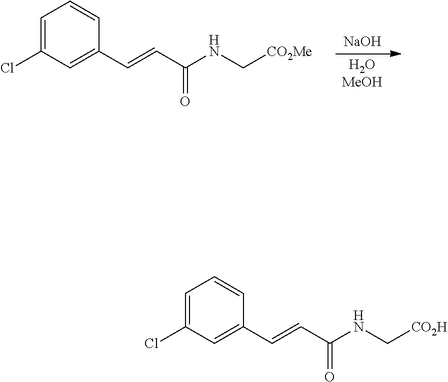 [SMILES: COC(=O)CNC(=O)/C=C/c1cccc(Cl)c1 ; "NaOH" ; O=C(O)CNC(=O)/C=C/c1cccc(Cl)c1 ; "H2OMeOH"]

To a solution of (E)-methyl 2-(3-(3-chlorophenyl)acrylamido)acetate (1.553 g, 6.14 mmol) in MeOH (10 mL) was added 2.0 N NaOH (6.2 mL, 12.4 mmol). The mixture was stirred at room temperature for 1 h, after which time analytical HPLC indicated that the reaction was complete. The mixture was acidified by with a 1N HCl solution. The solvent was removed in vacuo to obtain the crude which was used to the next step with no further purification. ESI-MS (m/z): 240, [M+1]⁺.

### Step 3. (E)-3-(3-chlorophenyl)-N-(2-((1,1-dioxidotetrahydro-2H-thiopyran-4-yl)(methyl)amino)-2-oxoethyl)acrylamide, ML264

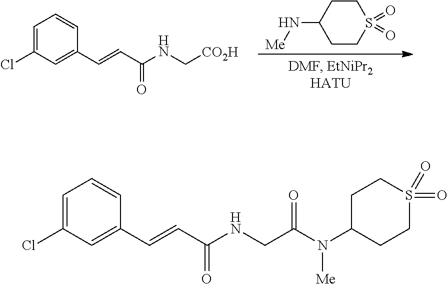

To a mixture of (E)-2-(3-(3-chlorophenyl)acrylamido)acetic acid (31.5 mg, 0.13 mmol) in DMF (1 mL) was added DIEA (52 mg, 0.4 mmol) and HATU (50 mg, 0.13 mmol). The mixture was stirred for 5 min, and then 4-(methylamino)tetrahydro-2H-thiopyran 1,1-dioxide hydrochloride (26 mg, 0.13 mmol) was added. The reaction mixture was stirred at room temperature for 30 min. The completion reaction was monitored by analytical HPLC. The solvent was removed in vacuo to obtain the crude which was purified by prep-HPLC (Acetonitrile/MeOH(1:1)/water 40˜100%) to obtain the title compound, 24.6 mg (49%). ESI-MS (m/z): 385.1, [M+1]+. 1H NMR (400 MHz, CD3CN) δ 7.63 (s, 1H), 7.51-7.52 (m, 1H), 7.48 (d, J=16.0 Hz, 1H), 7.39-7.41 (m, 2H), 6.91-7.02 (m, 1H), 6.78 (d, J=16.0 Hz, 1H), 4.65 (m, 1H), 4.08 (d, J=4.8 Hz, 2H, accompanied by smaller d in a 2.2:1 ratio at 4.22 due to amide isomerism), 3.19-3.35 (m, 2H), 2.99-3.04 (m, 2H), 2.87 (s, 3H, accompanied by smaller s at 2.83 in a 2.2:1 ratio due to amide isomerism), 2.04-2.39 (m, 4H).

Following procedures analogous to Example 1, the compounds 2-31, above, were prepared and their structures confirmed.

### Evaluations

Various structurally similar compounds were also tested for inhibition of KLF5 expression. As shown in Table 1, above, the inventors herein have ascertained certain structural limits to bioactivity. Compounds 24-31, having >100 μM IC₅₀ values are deemed to be inactive.

It is within ordinary skill using the procedures provided herein and in references cited herein, which are incorporated by reference in their entireties, to evaluate any compound disclosed and claimed herein for effectiveness for in vivo evaluation of antitumor activity as well as in the various cellular assays found in the scientific literature. Accordingly, the person of ordinary skill, using the disclosure of the present application in conjunction with the disclosures of documents cited herein, and the knowledge of the person of ordinary skill, can prepare and evaluate any of the claimed compounds for effectiveness as a potential human therapeutic agent, without undue experimentation.

Any compound found to be effective as an antitumor agent can likewise be further tested in animal models, and in human clinical studies, using the skill and experience of the investigator to guide the selection of dosages and treatment regimens.

### DOCUMENTS CITED

1. Ptashne M, Regulation of transcription: from lambda to eukaryotes. Trends Biochem Sci, 2005. 30 (6): p. 275-9. PMID: 15950866.
2. Fre S, Vignjevic D, Schoumacher M, Duffy S L, Janssen K P, Robine S, Louvard D. Epithelial morphogenesis and intestinal cancer: new insights in signaling mechanisms. Adv Cancer Res. 2008, 100:85-111.
3. Goldstein B G, Chao H H, Yang Y, Yermolina Y A, Tobias J W, Katz J P. Overexpression of Kruppel-like factor 5 in esophageal epithelia in vivo leads to increased proliferation in basal but not suprabasal cells. Am J Physiol Gastrointest Liver Physiol, 2007 292 (6), G1784-92.
4. McConnell B B, Ghaleb A M, Nandan M O, Yang V W. The diverse functions of Krüppel-like factors 4 and 5 in epithelial biology and pathobiology. Bioessays, 2007, 29(6):549-57. Erratum in: Bioessays. 2007, 29(9), 946.
5. Ghaleb A M, Nandan M O, Chanchevalap S, Dalton W B, Hisamuddin I M, Yang V W. Krüppel-like factors 4 and 5: the yin and yang regulators of cellular proliferation. Cell Res, 2005, 15(2), 92-6.
6. Yang Y, Tetreault M P, Yermolina Y A, Goldstein B G, Katz J P. Krüppel-like factor 5 controls keratinocyte migration via the integrin-linked kinase. J Biol Chem, 2008, 283(27), 18812-20.
7. Nandan M O, Yoon H S, Zhao W, Ouko L A, Chanchevalap S, Yang V W. Krüppel-like factor 5 mediates the transforming activity of oncogenic H-Ras. Oncogene, 2004, 23(19), 3404-13.
8. Nandan M O, McConnell B B, Ghaleb A M, Bialkowska A B, Sheng H, Shao J, Babbin B A, Robine S, Yang V W. Kruppel-like factor 5 mediates cellular transformation during oncogenic KRAS-induced intestinal tumorigenesis. Gastroenterology, 2008, 134,120.
9. McConnell B B, Bialkowska A B, Nandan M O, Ghaleb A M, Gordon F J, Yang V W. Haploinsufficiency of Kruppel-like factor 5 rescues the tumor-initiating effect of the ApcMin mutation in the intestine. Cancer Research, 2009, 69, 4125.
10. Nandan M O, Ghaleb A M, McConnell B B, Patel N V, Robine S, Yang V W. Kruppel-like factor 5 is a crucial mediator of intestinal tumorigenesis in mice harboring combined ApcMin and KRASV12 mutations. Molecular Cancer, 2010, 9, 63.
11. Chanchevalap S, Nandan M O, Merlin D, Yang V W. All-trans retinoic acid inhibits proliferation of intestinal epithelial cells by inhibiting expression of the gene encoding Kruppel-like factor 5. FEBS Lett, 2004, 578 (1-2), 99-105.
12. Sun R, Chen X, Yang V W. Intestinal-enriched Krüppel-like factor (Krüppel-like factor 5) is a positive regulator of cellular proliferation. J Biol Chem, 2001, 276(10), 6897-900.
13. Tong D, Czerwenka K, Heinze G, Ryffel M, Schuster E, Witt A, Leodolter S, Zeillinger R. Expression of KLF5 is a Prognostic Factor for Disease-Free Survival and Overall Survival in Patients with Breast Cancer. Clin Cancer Res, 2006, 12, 2442.
14. Rosen E D, MacDougald O A, Adipocyte differentiation from the inside out. Nature Rev Mol Cell Biol, 2006, 7, 885-896.
15. Li H, Miao S, Dong L, Shub Y, Shao D, Chen B, Han M, Zhang Y. Clinicopathological correlation of Krüppel-like factor 5 and matrix metalloproteinase-9 expression and cartilage degeneration in human osteoarthritis. Pathology—Research and Practice, 2012, 208, 9-14.
16. Lipinski C A, Lombardo F, Dominy B W, and Feeney P J, Experimental and computational approaches to estimate solubility and permeability in drug discovery and development settings. Advanced Drug Delivery Reviews, 2001, 46, 3-26.
17. Walters W P, Going further than Lipinski's rule in drug design. Expert Opinion on Drug Discovery, 2012, 7(2), 99-107.
18. Rishton G M, Nonleadlikeness and leadlikeness in biochemical screening. Drug Discovery Today, 2003, 8(2), 86-96.
19. Benigni R and Bossa C, Mechanisms of Chemical Carcinogenicity and Mutagenicity: A Review with Implications for Predictive Toxicology. Chem. Rev., 2011, 111(4), 2507-2536
20. Stepan A F, Walker D P, Bauman J, Price D A, Baillie T A, Kalgutkar A S, and Aleo M D, Structural Alert/Reactive Metabolite Concept as Applied in Medicinal Chemistry to Mitigate the Risk of Idiosyncratic Drug Toxicity: A Perspective Based on the Critical Examination of Trends in the Top 200 Drugs Marketed in the United States. Chem ResToxicol, 2011, 24, 1345-1410.
21. Bialkowska A, Crisp M, Bannister T, He Y, Chowdhury S, Schurer S, Chase P, Spicer T, Madoux F, Tian C, Hodder P, Zaharevitz D, Yang V W. Identification of Small-Molecule Inhibitors of the Colorectal Cancer Oncogene Kruppel-Like Factor 5 Expression by Ultrahigh-Throughput Screening. Molecular Cancer Therapeutics, 2011, 10, 2043-2051.
22. Chen C, Bhalala H V, Qiao H, Dong J T. A possible tumor suppressor role of the KLF5 transcription factor in human breast cancer. Oncogene 2002, 21, 6567-72.
23. Chen C, Zhou Y, Zhou Z, Sun X, Otto K B, Uht R M, Dong J T. Regulation of KLF5 involves the Sp1 transcription factor in human epithelial cells. Gene, 2004, 330, 133.

The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A KLF5 expression-inhibitory compound of formula (I) 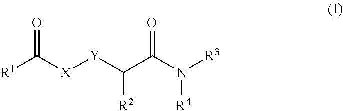
wherein
each R is independently H or (C₁-C₆)alkyl;
R¹ is aryl, arylalkenyl, heteroaryl, or heteroarylalkenyl, wherein any aryl or heteroaryl of R¹ is optionally mono- or independently multi-substituted with J;
X═CHR, O, or NR₂;
Y═CHR or absent;
R² is H, (C₁-C₆)alkyl, hydroxy(C₁-C₆)alkyl, (C₁-C₆)alkylNR₂, aryl, benzyl, p-hydroxybenzyl, (C₁-C₆)alkyl-C(═O)NR₂, (C₁-C₆)alkyl-C(═O)OR, or heteroaryl;
R³═H, (C₁-C₆)alkyl; (C₃-C₁₀)cycloalkyl, (C₁-C₆)alkylNR₂, (C₁-C₆)alkyl(C₃-C₁₀)cycloalkyl, or (C₁-C₆)alkyl-(3- to 10-membered heterocyclyl);
R⁴ is a 5-7 membered heterocyclyl comprising at least one C(═O)NR group or SO₂ group, optionally further comprising an additional NR group
J is halo, CF₃, OCF₃, CH₃, CH₂CH₃, SO₂R, SO₂NR₂, or C(═O)NR₂;
or a pharmaceutically acceptable salt thereof.

2. The compound of claim 1, wherein R¹ is a group of formula 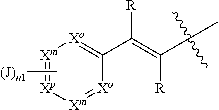
wherein
X^{o}, X^{m} and X^{p} are carbon or optionally any one or two independently selected X^{o}, X^{m}, or X^{p} is nitrogen;
R and J are as defined in claims 1; and n1 is 0, 1, 2, 3, 4, or 5, provided that when any of X^{o}, X^{m}, or X^{p} are nitrogen, J is absent therefrom; and,
a wavy line indicates a point of bonding.

3. The compound of claim 2 wherein all of X^{o}, X^{m}, and X^{p} are carbon bearing a respective hydrogen or J, and both R are hydrogen.

4. The compound of claim 1 wherein R¹ is of formula 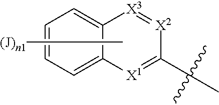
wherein
X¹, X² and X³ are each independently carbon or nitrogen;
the ring comprising X¹, X², and X³, or the aryl ring fused thereto, or both, can be substituted with n1 J group wherein J is halo, SO₂R, SO₂NR₂, or C(═O)NR₂ and n1 is 0, 1, 2, 3, 4, or 5; and,
a wavy line indicates a point of bonding.

5. The compound of claim 4 wherein R¹ is any one of 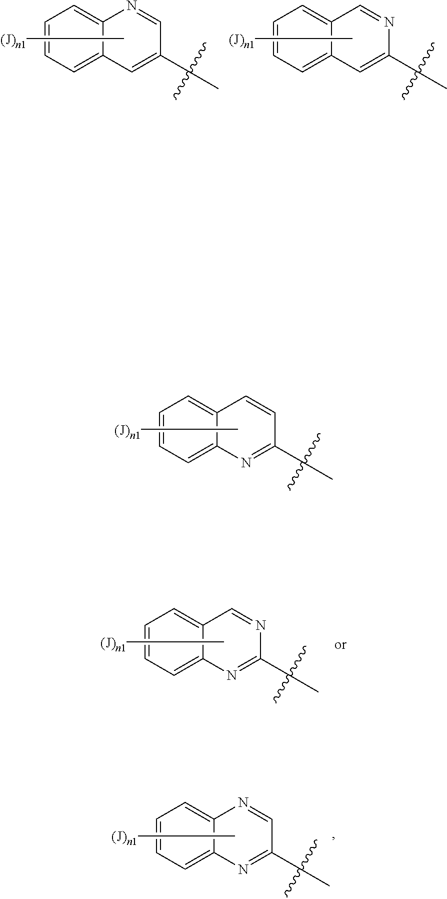
wherein J and n1 are as defined in claim 4.

6. The compound of claim 1 wherein R¹ is of formula 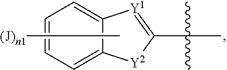
wherein
Y¹ is carbon or nitrogen, provided that when Y¹ is nitrogen, hydrogen and J are absent therefrom;
Y² is CR₂, NR, O, or S(O)q wherein q is 0, 1, or 2;
the ring comprising Y¹ and Y², or the aryl ring fused thereto, or both, can be substituted with n1 J group wherein J is halo, SO₂R, SO₂NR₂, or C(═O)NR₂ and n1 is 0, 1, 2, 3, 4, or 5.

7. The compound of claim 6 wherein R¹ is any one of 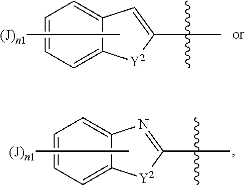
wherein Y², J, and n1 are as defined in claim 6.

8. The compound of claim 1 wherein X is NR₂ and Y is absent.

9. The compound of claim 1 wherein R² is H, methyl, hydroxymethyl, benzyl, or p-hydroxybenzyl.

10. The compound of claim 1 wherein R⁴ is any one of

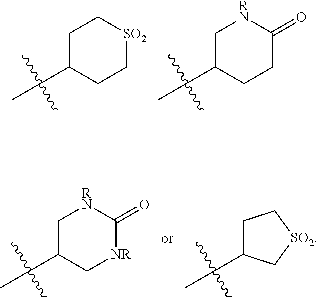

11. The compound of claim 1 selected from any one of

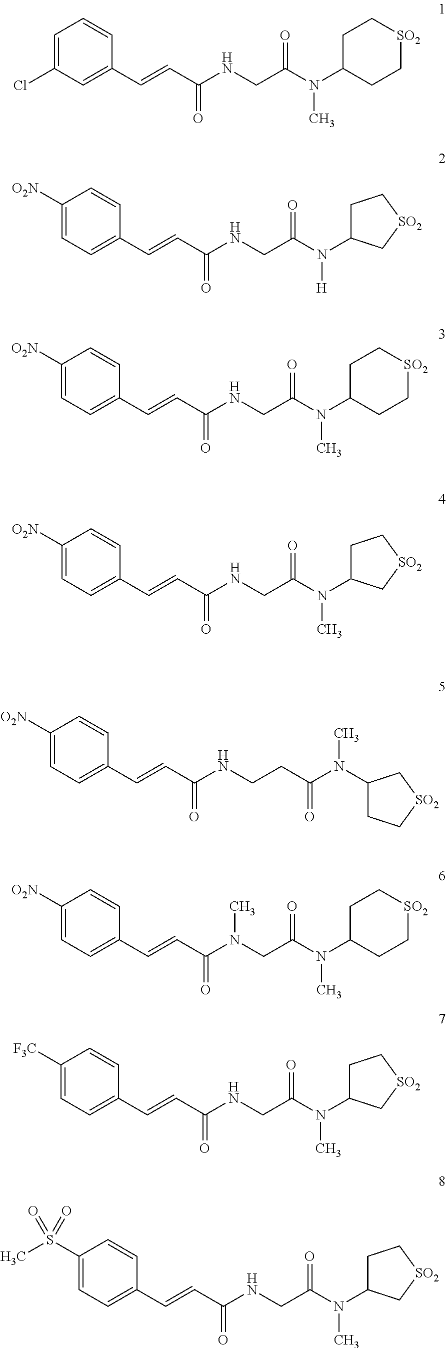

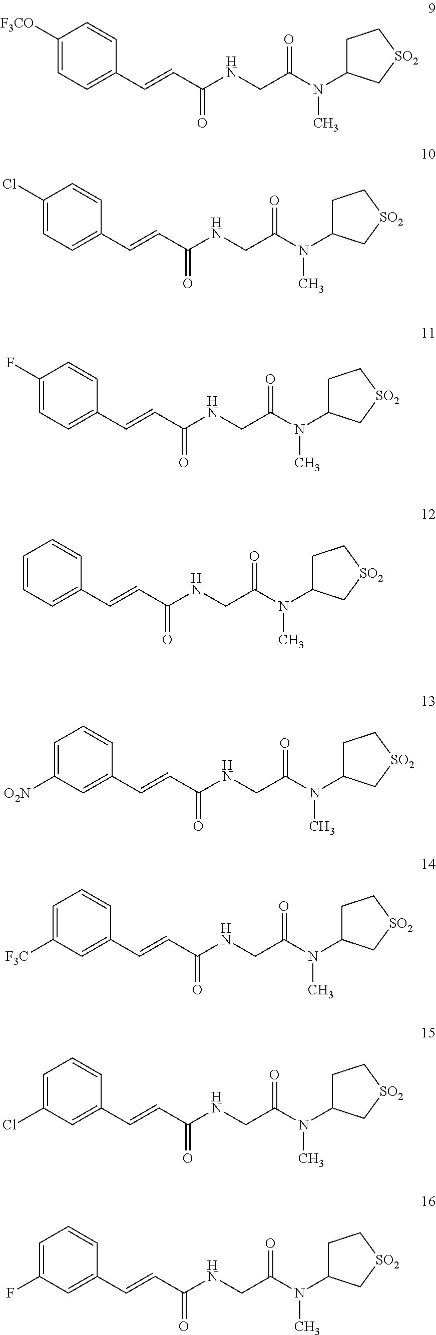

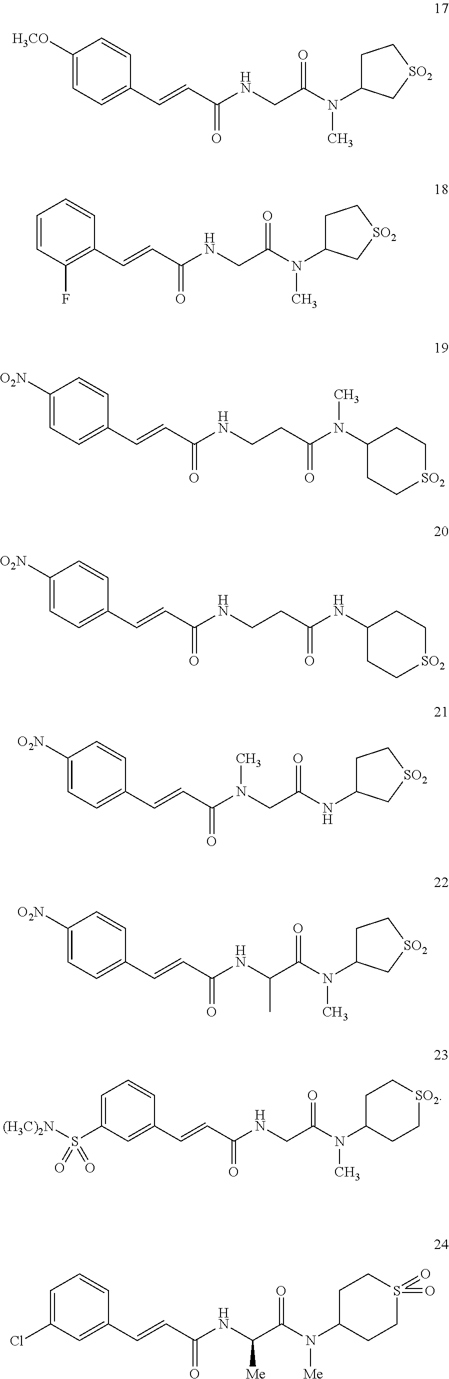

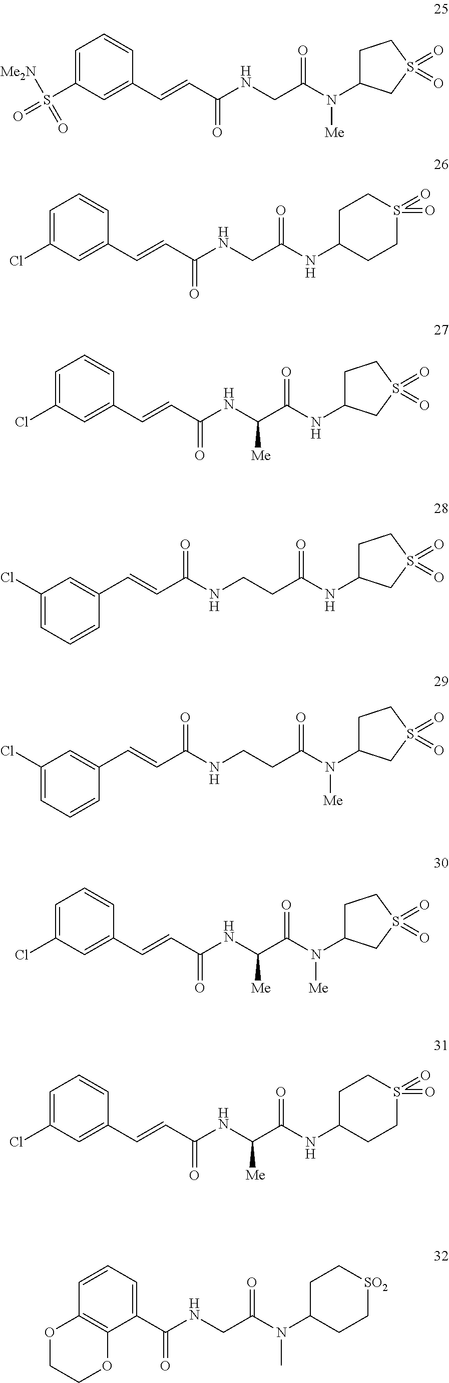

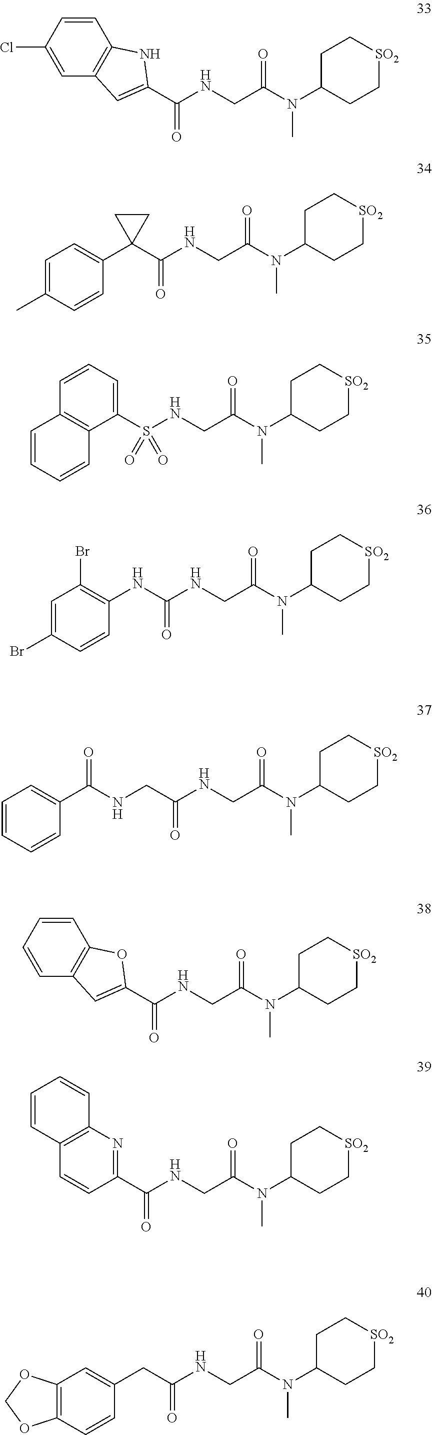

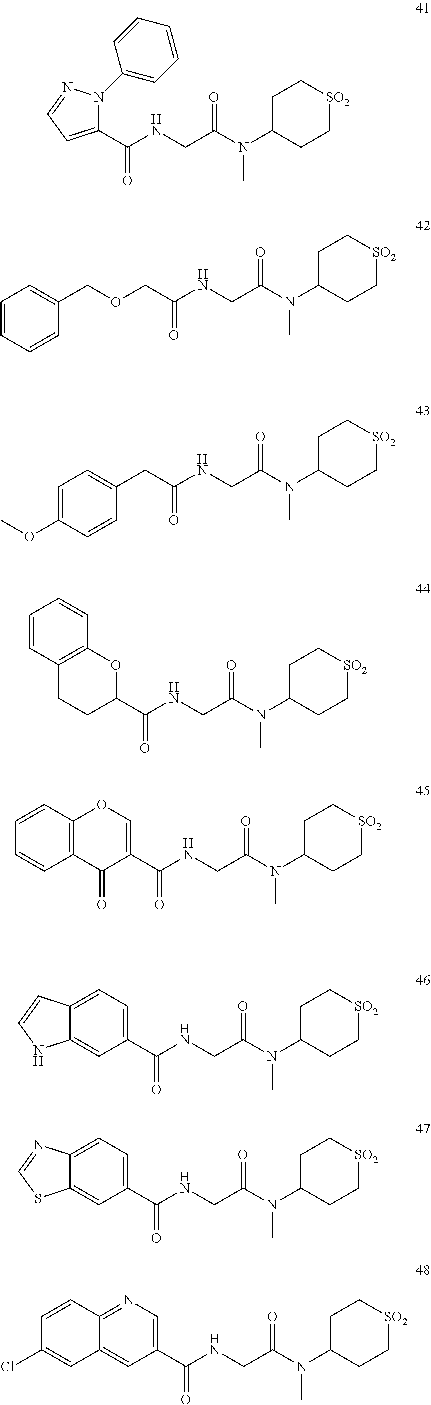

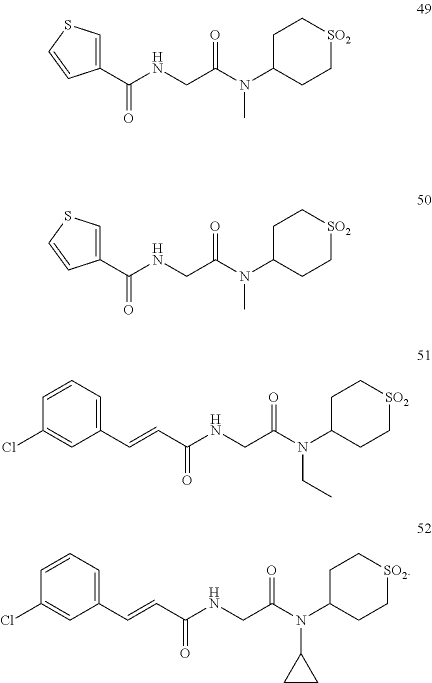 [SMILES: CN(C(=O)CNC(=O)c1ccsc1)C1CCS(=O)(=O)CC1 ; CN(C(=O)CNC(=O)c1ccsc1)C1CCS(=O)(=O)CC1 ; CCN(C(=O)CNC(=O)/C=C/c1cccc(Cl)c1)C1CCS(=O)(=O)CC1 ; O=C(/C=C/c1cccc(Cl)c1)NCC(=O)N(C1CC1)C1CCS(=O)(=O)CC1]

12. A pharmaceutical composition comprising a compound of claim 1 and a pharmaceutically acceptable excipient.

13. A pharmaceutical composition comprising a compound of claim 11, and a pharmaceutically acceptable excipient.

14. A method of treatment of a patient for control of a condition wherein inhibition of KLF5 expression is medically indicated, comprising administering an effective amount of a compound of claim 1 to the patient.

15. A method of treatment of a patient for control of a condition wherein inhibition of KLF5 expression is medically indicated, comprising administering an effective amount of any one compound of claim 11 to the patient.

16. The method of claim 14 to delay or prevent colon cancer onset, halt growth of existing tumors, and/or decrease reoccurrence.

17. The method of claim 14 wherein the condition comprises colorectal cancer, diabetes, obesity, lipid homeostasis, cardiovascular disease, or arthritis, or a combination thereof.

18. The method of claim 15 to delay or prevent colon cancer onset, halt growth of existing tumors, and/or decrease reoccurrence.

19. The method of claim 15 wherein the condition comprises colorectal cancer, diabetes, obesity, lipid homeostasis, cardiovascular disease, or arthritis, or a combination thereof.

20. (canceled)

21. (canceled)
